(19) United States
(12) Patent Application Publication
(10) Pub. No.: US 2008/0045580 A1
(43) Pub. Date: Feb. 21, 2008

(54) Novel Isoxazole Compounds Having Ppar Agonist Activity

(75) Inventors: Gurram Ranga Madhavan, Hyderabad (IN); Javed Iqbal, Hyderabad (IN); Debnath Bhuniya, Hyderabad (IN); Saibal Kumar Das, Hyderabad (IN); Sudhir Kumar Sharma, Hyderabad (IN); Ranjan Chakrabarti, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(73) Assignees: DR REDDY'S LABORATORIES LIMITED, HYDERBAD, ANDHRA PRADESH (IN); DR. REDDY'S LABORATORIES, INC., BRIDGEWATER, NJ (US)

(21) Appl. No.: 11/577,031

(22) PCT Filed: Oct. 11, 2005
(86) PCT No.: PCT/US05/36474

§ 371 Date: Apr. 11, 2007

(30) Foreign Application Priority Data
Oct. 11, 2004 (IN) 1051/CHE/2004

(51) Int. Cl. A61K 31/42; A61P 29/00; A61P 3/10; A61P 9/00; A61P 9/12; C07D 261/08; C07D 413/12

(52) U.S. Cl. 514/378; 548/247

(57) ABSTRACT

The present invention relates to novel isoxazole compounds of formula (I) having (PPAR) agonist activity, pharmaceutical compositions containing such compounds and methods for their use.

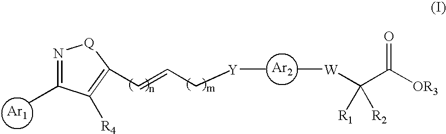

### CROSS-REFERENCE TO RELATED APPLICATIONS

This application claims priority to Indian Patent Application No. 1051/CHE/2004, filed on Oct. 11, 2004, the contents of which are incorporated by reference in their entirety.

### BACKGROUND

Peroxisome Proliferator Activated Receptors (PPARs) are orphan receptors belonging to the steroid/retinoid receptor super family of ligand activated transcription factors. Three mammalian Peroxisome Proliferator Activated Receptors (PPARs) have been isolated and termed PPARα, PPARγ and PPARδ. These PPARs are believed to regulate expression of target genes by binding to DNA sequence elements.

Certain PPAR agonist compounds are believed to be useful candidates for treatment of metabolic disorders. See, e.g., U.S. Pat. Nos. 5,885,997 and 6,054,453, and U.S. Publication No. 2003/0229083. Nevertheless, there exists a continuing need for new PPAR agonist compounds.

### SUMMARY

In accordance with one aspect, the invention provides a derivative, which is a compound and/or a pharmaceutically acceptable salt thereof, wherein said compound has the formula (I): 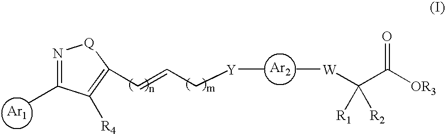
wherein Ar₁ is an optionally substituted aryl or heteroaryl;
Ar₂ is an optionally substituted aryl;
R₁ and R₂, which may be the same or different, are independently hydrogen, hydroxy, halogen or an optionally substituted alkyl, cycloalkyl, aryl, aralkyl, aryloxy, heteroaryl, heterocyclyl or heteroaralkyl, or R₁ and R₂ together form an optionally substituted 5 or 6 membered cyclic ring, which optionally contains one or two hetero atoms selected from O, S or N; R₃ and R₄, which may be the same or different, are independently hydrogen or an optionally substituted alkyl, cycloalkyl, aryl, aralkyl, heteroaryl, heterocyclyl or heteroaralkyl;
Y is O, S, CH₂ or NR₅, wherein R₅ is hydrogen, alkyl or cycloalkyl;
W is O, S or CH₂;
Q is O or S;
m is 0-6; and
n is 0-1.

In accordance with another aspect, the invention provides a pharmaceutical composition comprising a derivative, which is a compound and/or a pharmaceutically acceptable salt thereof, wherein said compound has the formula (I), and one or more pharmaceutically acceptable excipients.

In accordance with another aspect, the invention provides a method for producing a PPAR-α agonist activity in an individual in need of such activity comprising administering to said individual a therapeutically effective amount of a pharmaceutical composition comprising a derivative, which is a compound and/or a pharmaceutically acceptable salt thereof, wherein said compound has the formula (I), and one or more pharmaceutically acceptable excipients.

In accordance with another aspect, the invention provides a method for treating a disease or disorder in which PPAR-α agonist activity is desired comprising administering to an individual in need of such treatment a therapeutically effective amount of a pharmaceutical composition comprising a derivative, which is a compound and/or a pharmaceutically acceptable salt thereof, wherein said compound has the formula (I), and one or more pharmaceutically acceptable excipients.

The compounds of formula (I) are useful in reducing body weight and for the treatment and/or prophylaxis of diseases such as atherosclerosis, stroke, peripheral vascular diseases and related disorders. These compounds are useful for the treatment and/or prophylaxis of hyperlipidemia, hyperglycemia, hypercholesterolemia, lowering of atherogenic lipoproteins, VLDL (very low density lipoprotein) and LDL (low density lipoprotein). The compounds of the present invention can be used for the treatment of renal diseases including glomerulonephritis, glomerulosclerosis, nephrotic syndrome, hypertensive nephrosclerosis and nephropathy. The compounds of formula (I) are also useful for the treatment and/or prophylaxis of leptin resistance, impaired glucose tolerance, disorders related to syndrome X such as hypertension, obesity, insulin resistance, coronary heart disease and other cardiovascular disorders. These compounds may also be useful for improving cognitive functions in dementia, treatment and/or prophylaxis of diabetes, diabetic complications, disorders related to endothelial cell activation, psoriasis, polycystic ovarian syndrome (PCOS), inflammatory bowel diseases, osteoporosis, myotonic dystrophy, pancreatitis, arteriosclerosis, retinopathy, xanthoma, eating disorders, inflammation and for the treatment of cancer. The compounds of the present invention are also useful in the treatment and/or prophylaxis of the above said diseases in combination/concomittant with one or more of a HMG CoA reductase inhibitor; a cholesterol absorption inhibitor; an antiobesity drug; a lipoprotein disorder treatment drug; a hypoglycemic agent; insulin; a biguanide; a sulfonylurea; thiazolidinedione; a dual PPARα and γ agonist or a mixture thereof.

### DETAILED DESCRIPTION OF EMBODIMENTS

To describe the invention, certain terms are defined herein as follows.

The use of singular includes the use of plural. In a non-limiting example, a recitation of “a derivative” includes a single derivative, as well as multiple derivatives.

The term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless whether it exists in a free form or and an associated forms.

The term “stereoisomers” is used to refer to both optical isomers and geometrical isomers. A recitation of the chemical structure of the compound encompasses all structural variations possible within the structure as shown.

Thus, some of the described compounds have optical centers. If the optical configuration at a given optical center is not defined with specificity, the recitation of chemical structure covers all optical isomers produced by possible configurations at the optical center. The term “optical isomer” defines a compound having a defined optical configuration at least one optical center. This principle applies for each structural genus described herein, as well as for each subgenus and for individual structures. For example, the recitation of a molecular portion as 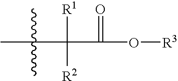
encompasses optical isomers with R and S configurations at the optical center (which arises when R¹ and R² are not identical):

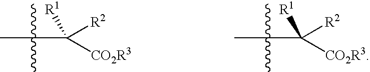

For the purpose of additional illustration, for example, the recitation “a compound of the structure 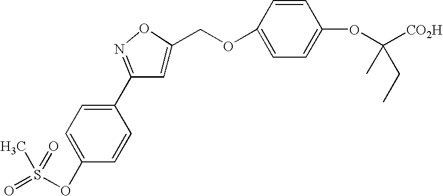
generically encompasses both enantiomers individually: 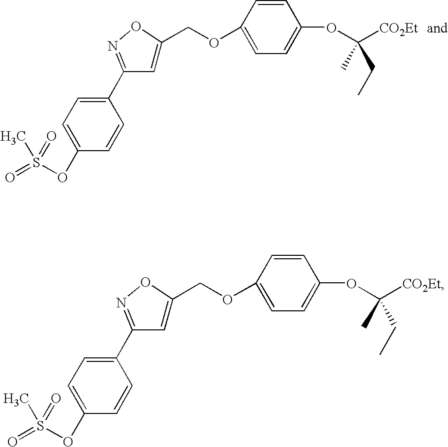
as well as the racemic mixture.

The individual optical isomers may be obtained by using reagents in such a way to obtain single isomeric form in the process wherever applicable or by conducting the reaction in the presence of reagents or catalysts in their single enantiomeric form. Some of the preferred methods of resolution of racemic compounds include use of microbial resolution, resolving the diastereomeric salts, amides or esters formed with chiral acids such as mandelic acid, camphorsulfonic acid, tartaric acid, lactic acid, and the like wherever applicable or chiral bases such as brucine, cinchona alkaloids and their derivatives and the like. Commonly used methods are compiled by Jaques et al. in “Enantiomers, Racemates and Resolution” (Wiley Interscience, 1981). Where appropriate the compounds of formula (I) may be resolved by treating with chiral amines, aminoacids, aminoalcohols derived from aminoacids; conventional reaction conditions may be employed to convert acid into an amide; the diastereomers may be separated either by fractional crystallization or chromatography and the stereoisomers of compound of formula (I) may be prepared by hydrolyzing the pure diastereomeric amide, ester or salt.

Some of the described compounds may exist as geometrical isomers (e.g., (E), (Z), etc.). If the geometrical configuration is not self-evident from the structure shown, the recitation of the structure generically covers all possible geometrical isomers. This principle applies for each structural genus described herein, as well as for each subgenus and for individual structures.

The compounds may form salts. The term “derivative” is used as a common term for the compound and its salts. Thus, the claim language “a derivative, which is a compound and/or a pharmaceutically-acceptable salt of said compound” is used to define a genus that includes any form of the compound of the given chemical structure and the salts of the recited compound. The use of the term “and/or” is intended to indicate that, for a compound of a given chemical structure, a claim to a “derivative” covers the compound individually, all of its salts individually, and the mixtures of compounds and the salt(s).

The term “pharmaceutically-acceptable salts” is intended to denote salts that are suitable for use in human or animal pharmaceutical products. The use of the term “pharmaceutically-acceptable” is not intended to limit the claims to substances (“derivatives”) found only outside of the body. Representative salts include, but are not limited to, Li, Na, K, Ca, Mg, Fe, Cu, Zn, Mn; N,N′-diacetylethylenediamine, betaine, caffeine, 2-diethylaminoethanol, 2-dimethylaminoethanol, N-ethylmorpholine, N-ethylpiperidine, glucamine, glucosamine, hydrabamine, isopropylamine, methylglucamine, morpholine, piperazine, piperidine, procaine, purines, theobromine, triethylamine, trimethylamine, tripropylamine, tromethamine, diethanolamine, meglumine, ethylenediamine, N,N′-diphenylethylenediamine, N,N′-dibenzylethylenediamine, N-benzyl phenylethylamine, choline, choline hydroxide, dicyclohexylamine, metformin, benzylamine, phenylethylamine, dialkylamine, trialkylamine, thiamine, aminopyrimidine, aminopyridine, purine, spermidine; alkylphenylamine, glycinol, phenyl glycinol; glycine, alanine, valine, leucine, isoleucine, norleucine, tyrosine, cystine, cysteine, methionine, proline, hydroxy proline, histidine, ornithine, lysine, arginine, serine, threonine, phenylalanine; unnatural amino acids; D-isomers or substituted amino acids; guanidine, substituted guanidine wherein the substituents are selected from nitro, amino, alkyl, alkenyl, alkynyl, ammonium or substituted ammonium salts and aluminum salts; sulphates, nitrates, phosphates, perchlorates, borates, hydrohalides, acetates, tartrates, maleates, citrates, succinates, oxalates, palmoates, methanesulphonates, benzoates, salicylates, hydroxynaphthoates, benzenesulfonates, ascorbates, glycerophosphates, or ketoglutarates. Salts encompassed within the term “pharmaceutically acceptable salts” can generally be prepared by reacting the free acid with a suitable organic or inorganic base.

The term “prodrug” is used to refer to a compound (and/or its salt) capable of converting, either directly or indirectly, into compounds described herein by the action of enzymes, gastric acid and the like under in vivo physiological conditions (e.g., enzymatic oxidation, reduction and/or hydrolysis).

In describing the compounds, certain nomenclature and terminology is used throughout to refer to various groups and substituents. These terms apply regardless of whether a term is used by itself or in combination with other terms. For example, the definition of “alkyl” applies to “alkyl” as well as to the “alkyl” portions of “alkoxy”, “alkylamino” etc. The description “Cₓ-C_{y}” refers to a chain of carbon atoms or a carbocyclic skeleton containing from x to y atoms, inclusive. The designated range of carbon atoms may refer independently to the number of carbon atoms in the chain or the cyclic skeleton, or to the portion of a larger substituent in which the chain or the skeleton is included. For example, the recitation “(C₁-C₅) alkyl” refers to an alkyl group having a carbon chain of 1 to 5 carbon atoms, inclusive of 1 and 5. The chains of carbon atoms of the groups and substituents described and claimed herein may be saturated or unsaturated, straight chain or branched, substituted or unsubstituted.

The description 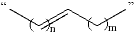
refers to a linkage between n double bonds and m single bonds. Thus, for example, when n is 0 and m is 1, the linkage between “ . . . ” is —CH₂—; when n is 0 and m is 2, the linkage between “ . . . ” is —CH₂—CH₂—; when n is 0 and m is 3, the linkage between “ . . . ” is —CH₂—CH₂—CH₂—; and when n is 1 and m is 1, the linkage between “ . . . ” is —CH═CH—CH₂—.

“Alkyl” means an aliphatic hydrocarbon group, which may be straight or branched and comprising about 1 to about 24 carbon atoms in the chain. Branched means that one or more lower alkyl groups such as methyl, ethyl or propyl, are attached to a linear alkyl chain. “Lower alkyl” means an alkyl group having about 1 to about 6 carbon atoms in the chain, which may be straight or branched. The alkyl group can be optionally substituted by replacing an available hydrogen on the chain with one or more substituents, which may be the same or different. Non-limiting examples of suitable alkyl groups include methyl, ethyl, n-propyl, isopropyl, n-butyl, and t-butyl.

“Aryl” means an aromatic monocyclic or multicyclic ring system comprising about 6 to about 14 carbon atoms, preferably about 6 to about 10 carbon atoms. Non-limiting examples of suitable aryl groups include phenyl and naphthyl. The aryl group can be optionally substituted by replacing an available hydrogen on the ring with one or more substituents, which may be the same or different. The “aryl” group can also be substituted by linking two adjacent carbons on its aromatic ring via a combination of one or more carbon atoms and one or more oxygen atoms such as, for example, methylenedioxy, ethylenedioxy, and the like.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. The “heteroaryl” can be optionally substituted by replacing an available hydrogen on the ring by one or more substituents, which may be the same or different. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrrolyl, triazolyl, and the like.

“Cycloalkyl” means a non-aromatic mono- or multicyclic ring system comprising about 3 to about 10 carbon atoms. The cycloalkyl can be optionally substituted by replacing an available hydrogen on the ring by one or more substituents, which may be the same or different. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Heterocyclyl” means a non-aromatic saturated monocyclic or multicyclic ring system comprising about 3 to about 10 ring atoms in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. The heterocyclyl can be optionally substituted by replacing an available hydrogen on the ring by one or more substituents, which may be the same or different. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, pyranyl, tetrahydrothiophenyl, morpholinyl and the like.

“Heteroarylalkyl” means a heteroaryl-alkyl-group in which the heteroaryl and alkyl are as previously described. Non-limiting examples of suitable heteroarylalkyl groups include pyridylmethyl, 2-(furan-3-yl)ethyl and quinolin-3-ylmethyl. The bond to the parent moiety is through the alkyl.

“Halo” means fluoro, chloro, bromo or iodo groups.

“Halogen” means fluorine, chlorine, bromine or iodine.

“Haloalkyl” means an alkyl as defined above wherein one or more hydrogen atoms on the alkyl is replaced by a halo group defined above.

“Alkoxy” means an alkyl-O— group in which the alkyl group is as previously described. Non-limiting examples of suitable alkoxy groups include methoxy, ethoxy, n-propoxy and isopropoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Alkylsulfonyloxy” means —OS(O)₂-alkyl group in which the alkyl group is as previously described. Non-limiting examples of suitable alkylsulfonyloxy groups include, but are not limited to methylsulfonyloxy, trifluoromethylsulfonyloxy, propylsulfonyloxy, and the like. The bond to the parent moiety is through the ether oxygen.

“Alkylsulfonylamino” means the —NS(O)₂-alkyl group in which the alkyl group is as previously described. Non-limiting examples of suitable alkylsulfonylamino groups include, but are not limited to, methylsulfonylamino, propylsulfonylamino, and the like. The bond to the parent moiety is through the nitrogen.

The term “optionally substituted” means optional substitution with one or more groups, radicals or moieties (i.e., “substituents”), which can be the same or different. Representative substituents include, but are not limited to, halo, alkyl, alkoxy, haloalkyl, aryl, aryloxy, alkylsulfonyloxy, hydroxy, amino, alkylsulfonylamino, nitro, and carboxy. The optional substituents themselves can be optionally substituted and contain one or more hetero atoms.

An embodiment of the present invention provides preparation of the novel compounds of formula (I) according to the procedure of the following schemes, using appropriate materials. Those skilled in the art will readily understand that known variations of the conditions and processes of the following preparative procedures can be used to prepare these compounds. Moreover, by utilizing the procedures described in detail, one of ordinary skill in the art can readily prepare additional compounds of the present invention claimed herein. All temperatures are in degrees Celsius unless otherwise noted.

According to an embodiment of the present invention, the compound of general formula (I) where Y represents O or S, m represents 0-6, n represents 0-1, and all other symbols are as defined earlier, can be prepared by the process as shown in Scheme-I below (the double bond “n” is shown only in compound (Ia) as a matter of convenience):

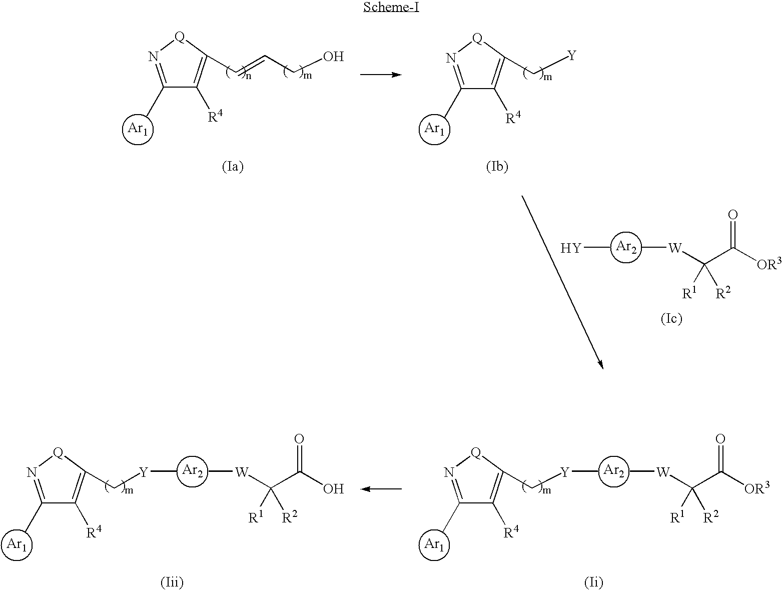

The compound of formula (Ia) was converted to a compound of formula (Ib) in the presence of mesylchloride, tosylchloride or C(Hal)₄, wherein “Hal” represents a halogen atom. The reaction was carried out in the presence of triphenylphosphine and the like. The solvent used in the reaction can be selected from dichloromethane, tetrahydrofuran, chloroform, dimethylether, diethylether, dioxane, benzene, toluene or mixtures thereof. The reaction was carried out at 25-30° C. The duration of the reaction can be in the range of 0.5 to 2 hours, preferably 0.5 to 1 hour. The temperature of the reaction can be maintained in the range of temperature range of −25 to 30° C., preferably 0° C. to 30° C.

The reaction of compound of formula (Ib), where L represents a leaving group such as halogen atom, p-toluenesulfonate, methanesulfonate, trifluoromethane sulfonate and the like, with a compound of formula (Ic) where all symbols are as defined earlier to produce a compound of the formula (Ii), where Y represents O or S, m represents 0-6, n represents 0-1, and R³ represents all the groups as defined earlier except hydrogen, can be carried out in the presence of aprotic solvents such as toluene, benzene, xylene, tetrahydrofuran, dimethylormamide, dimethylsulphoxide, dimethoxyethane, acetonitrile and the like. The reaction can be carried out in an inert atmosphere that can be maintained by using inert gases such as nitrogen, argon, helium and the like. The base used in the reaction can be selected from alkalis like sodium hydroxide, potassium hydroxide and the like; alkali metal carbonates such as sodium carbonate, potassium carbonate and the like; alkali metal hydrides such as sodium hydride, potassium hydride and the like; organometallic bases like n-butyl lithium, lithium diisopropylamide and the like; alkali metal amides like sodamide, organic base like triethyl amine, lutidine, collidine and the like. Acetone or acetonitrile can be used as solvent when alkali metal carbonate is used as a base. Phase transfer catalyst used can be selected from tetraalkyl ammoniumhalide, hydrogensulphate and the like. Additives used in the reaction can be alkali metal halides such as lithiumbromide and the like. The reaction temperature can be in the range of 0° C. to 160° C., preferably at a temperature in the range of 25 to 100° C. The duration of the reaction can be in the range from 1 to 120 hours, preferably from 2 to 24 hours.

The compound of general formula (Iii) where R³ represents hydrogen atom, Y represents O or S, m represents 0-6, n represents 0-1, and all other symbols are as defined earlier, can be prepared from a compound of formula (Ii), by hydrolysis using conventional methods. The base used in the reaction can be selected from sodium hydroxide, potassium hydroxide, lithium hydroxide, potassium carbonate, sodium carbonate and the like. The solvent used can be selected from alcohols such as methanol, ethanol, propanol, isopropanol and the like or mixtures thereof, water, tetrahydrofuran, dioxane, ether and the like or mixtures thereof. The temperature of the reaction can be in the range of 30 to 80° C., preferably at 25 to 30° C. The duration of the reaction can be in the range of 2 to 24 hours, preferably 2 to 12 hours.

According to another embodiment of the present invention, there is provided a process for the preparation of compound of formula (I), where m represents 3, n represents 0, Y represents O or S and all other symbols are as defined earlier, which comprises:

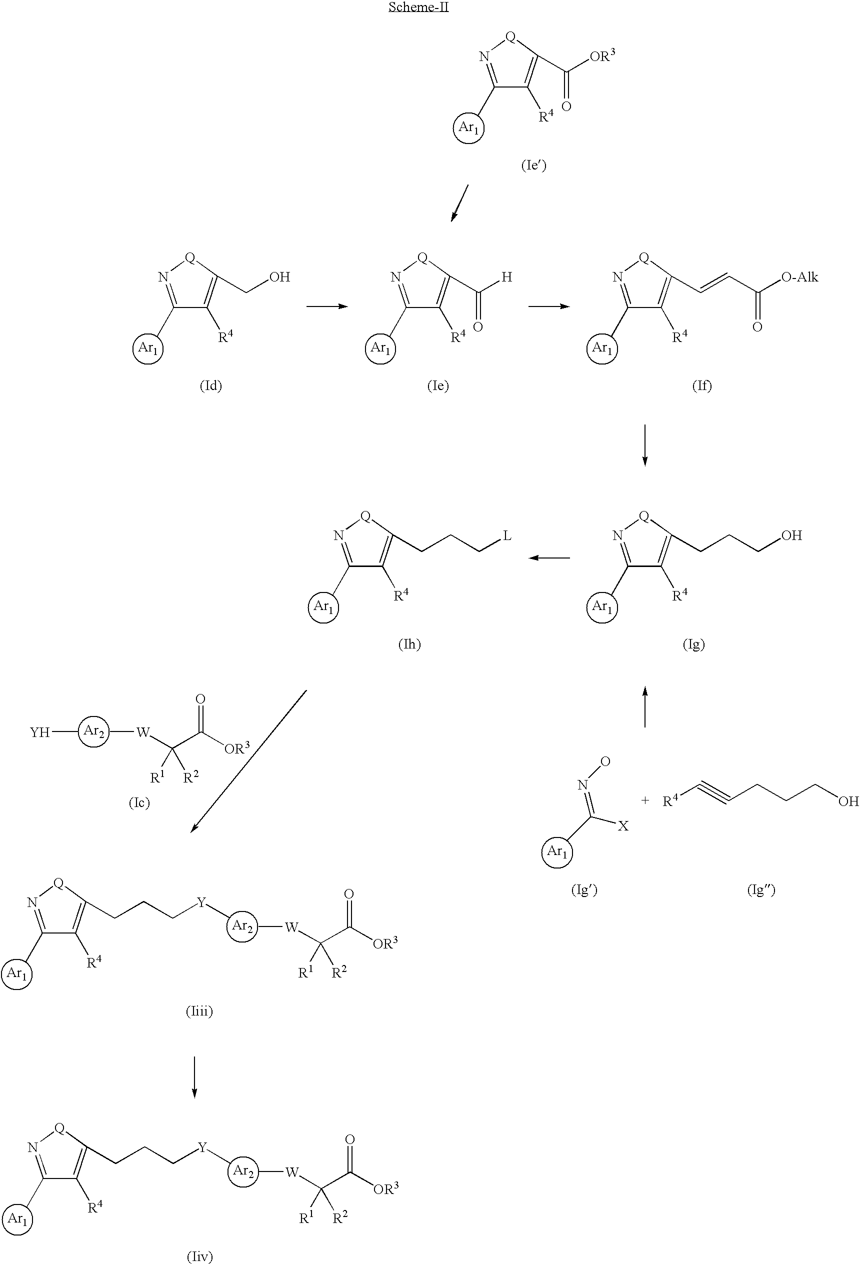

The compound of formula (Id) was converted to a compound of formula (Ie) by Swern oxidation or by using oxidizing agents like oxallyl chloride, manganese dioxide, chromium trioxide, PCC/PDC and the like. The solvent used in the reaction can be selected from DMSO, DCM, THF, chloroform and the like. The temperature of the reaction can be maintained in the range of −70 to 35° C. The duration of the reaction can be in the range of 1 to 24 hours.

The conversion of compound of formula (Ie′) may be converted to a compound of formula (Ie) by using a reagent like DIBAL.

The conversion of (Ie) to (If) was carried our by using triethylphosphoacetate under Wittig-Horner reaction conditions.

The compound of formula (Ig) was converted to a compound of formula (Ih) in the presence of mesylchloride, tosylchloride, SOCl₂, or C(Hal)₄, wherein “Hal” represents a halogen atom. The reaction was carried out in the presence of a reagent such as triphenylphosphine and the like. The solvent used in the reaction can be selected from dichloromethane, tetrahydrofuran, chloroform, dimethylether, diethylether, dioxane, benzene, toluene or mixtures thereof. The reaction can be carried out at 0 to 30° C. The duration of the reaction can be in the range of 0.5 to 2 hours, preferably 0.5 to 1 hour. The temperature of the reaction can be maintained in the range of temperature range of −25 to 30° C., preferably 0° C. to 30° C.

The reaction of compound of formula (Ih), where L represents a leaving group such as halogen atom, p-toluenesulfonate, methanesulfonate, trifluoromethane sulfonate and the like, with a compound of formula (Ic) where all symbols are as defined earlier to produce a compound of the formula (Iiii), where Y represents O or S, R³ represents all the groups as defined earlier except hydrogen, can be carried out in the presence of aprotic solvents such as toluene, benzene, xylene, tetrahydrofuran, dimethylormamide, dimethylsulphoxide, dimethoxyethane, acetonitrile and the like. The reaction was carried out in an inert atmosphere that can be maintained by using inert gases such as nitrogen, argon, helium and the like. The base used in the reaction can be selected from alkalis such as sodium hydroxide, potassium hydroxide and the like; alkali metal carbonates such as sodium carbonate, potassium carbonate and the like; alkali metal hydrides such as sodium hydride, potassium hydride and the like; organometallic bases like n-butyl lithium, lithium diisopropyl-amide and the like; alkali metal amides like sodamide, organic base like triethyl amine, lutidine, collidine and the like or mixtures thereof. Acetone can be used as solvent when alkali metal carbonate is used as a base. Phase transfer catalyst such as tetraalkyl ammoniumhalide, hydrogensulphate and the like was added. Additives used in the reaction can be alkali metal halides like lithiumbromide. The reaction temperature can be in the range of 0° C. to 160° C., preferably at a temperature in the range of 25 to 100° C. The duration of the reaction can be in the range of 1 to 120 hours, preferably from 2 to 24 hours.

The compound of general formula (Iiv) where R³ represents hydrogen atom, Y represents O or S and all other symbols are as defined earlier, was prepared from a compound of formula (Iiii), by hydrolysis using conventional methods. The base used in the reaction can be selected from sodium hydroxide, potassium hydroxide, lithium hydroxide, potassium carbonate, sodium carbonate and the like. The solvent used can be selected from alcohols such as methanol, ethanol, propanol, isopropanol and the like or mixtures thereof, water, tetrahydrofuran, dioxane, ether and the like or mixtures thereof. The temperature of the reaction may be in the range of 30 to 80° C., preferably at 25 to 30° C. The duration of the reaction can be in the range of 2 to 24 hours, preferably 2 to 12 hours.

The compound of formula (Ig) may also be prepared by reacting compound of formula (Ig′) with a compound of formula (Ig″).

According to another embodiment of the present invention, there is provided a process for the preparation of compound of formula (I), Y represents O or S and all other symbols are as defined earlier (the double bond “n” is not shown as a matter of convenience), which comprises:

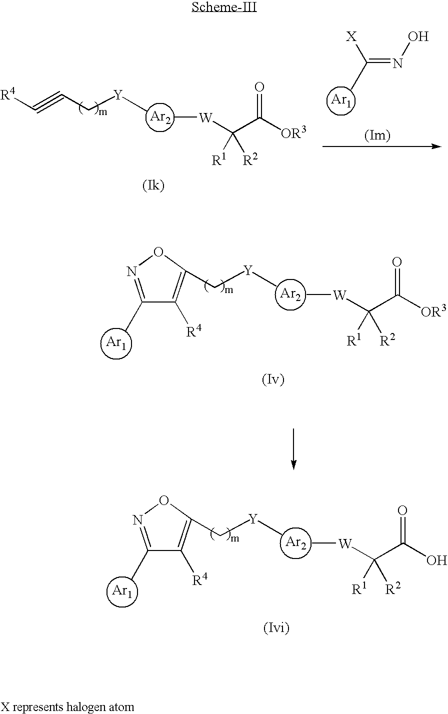

The compound (Ik) was reacted with compound of formula (Im) to obtain (Iv) in the presence of a base such as NEt₃, diisopropylamine, K₂CO₃ and the like. The solvent used in the reaction can be selected from chloroform, DCM, DCE, THF and the like. The temperature of the reaction can be maintained in the range of 0 to 90° C., preferably 20 to 35° C. The duration of the reaction can be maintained in the range of 3 to 4 hours.

The compound of general formula (Iv) where R³ represents hydrogen atom, Y represents O or S and all other symbols are as defined earlier, was prepared from a compound of formula (Iiv), by hydrolysis using conventional methods. The base used in the reaction can be selected from sodium hydroxide, potassium hydroxide, lithium hydroxide, potassium carbonate, sodium carbonate and the like. The solvent used can be selected from alcohols such as methanol, ethanol, propanol, isopropanol and the like or mixtures thereof, water, tetrahydrofuran, dioxane, ether and the like or mixtures thereof. The temperature of the reaction can be in the range of 30 to 80° C., preferably at 25 to 30° C. The duration of the reaction can be in the range of 2 to 24 hours, preferably 2 to 12 hours.

It is appreciated that in any of the above-mentioned reactions, any reactive group in the substrate molecule may be protected according to conventional chemical practice. Suitable protecting groups in any of the above mentioned reactions are tertiarybutyldimethylsilyl, methoxymethyl, triphenyl methyl, benzyloxycarbonyl, tetrahydropyran etc, to protect hydroxyl or phenolic hydroxy group; N-tert-butoxycarbonyl, N-benzyloxycarbonyl (N-Cbz), N-9-fluorenyl methoxy carbonyl (—N-FMOC), benzophenoneimine, propargyloxy carbonyl etc, for protection of amino or anilino group, acetal protection for aldehyde, ketal protection for ketone and the like. The methods of formation and removal of such protecting groups are those conventional methods appropriate to the molecule being protected.

The compounds of the present invention are administered to an individual in therapeutically effective amounts effective to agonize a PPAR where such treatment is needed, as, for example, in the prevention or treatment of diabetes, hypertension, coronary heart disease, atherosclerosis, stroke, peripheral vascular diseases, psoriasis, polycystic ovarian syndrome (PCOS), inflammatory bowel diseases, osteoporosis, myotonic dystrophy, pancreatitis, retinopathy, arteriosclerosis, xanthoma and related disorders.

The terms “individual,” “subject,” and “patient” refer to any subject for whom diagnosis, treatment, or therapy is desired. In one embodiment, the individual, subject, or patient is a human. Other subjects may include animals including, but not limited to cattle, sheep, horses, dogs, cats, guinea pigs, rabbits, rats, primates, opossums and mice. Other subjects include species of bacteria, phages, cell cultures, viruses, plants and other eucaryotes, prokaryotes and unclassified organisms.

The terms “treatment,” “treating,” “treat,” and the like are used herein to refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a human, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

The term “therapeutically effective amount” refers to the amount of a drug or pharmaceutical agent that will elicit the biological or medical response of a tissue, system or patient that is being sought.

The compounds described herein are typically administered in admixture with one or more pharmaceutical acceptable excipients or carriers in the form of a pharmaceutical composition. A “composition” may contain one compound or a mixture of compounds. A “pharmaceutical composition” is any composition useful or potentially useful in producing physiological response in a subject to which such pharmaceutical composition is administered. The term “pharmaceutically acceptable,” with respect to an excipient, is used to define non-toxic substances generally suitable for use in human or animal pharmaceutical products. The pharmaceutical composition may be in the forms normally employed, such as tablets, capsules, powders, syrups, solutions, suspensions and the like, may contain flavorants, sweeteners, etc., in suitable solid or liquid carriers or diluents, or in suitable sterile media to form injectable solutions or suspensions. Such compositions typically contain from 0.1 to 50%, preferably 1 to 20% by weight of active compound, the remainder of the composition being pharmaceutically acceptable carriers, diluents or solvents.

Suitable pharmaceutically acceptable carriers include solid fillers or diluents and sterile aqueous or organic solutions. The active ingredient will be present in such pharmaceutical compositions in the amounts sufficient to provide the desired dosage in the range as described above. Thus, for oral administration, the active ingredient can be combined with a suitable solid or liquid carrier or diluent to form capsules, tablets, powders, syrups, solutions, suspensions and the like. The pharmaceutical compositions, may, if desired, contain additional components such as flavourants, sweeteners, excipients and the like. For parenteral administration, the active ingredient can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or salts with base of the compounds. Aqueous solutions with the active ingredient dissolved in polyhydroxylated castor oil may also be used for injectable solutions. The injectable solutions prepared in this manner can then be administered intravenously, intraperitoneally, subcutaneously, or intramuscularly, with intramuscular administration being preferred in humans.

For nasal administration, the preparation may contain the active ingredient of the present invention dissolved or suspended in a liquid carrier, in particular an aqueous carrier, for aerosol application. The carrier may contain additives such as solubilizing agents, such as propylene glycol, surfactants, absorption enhancers such as lecithin (phosphatidylcholine) or cyclodextrin or preservatives such as parabenes.

Tablets, dragees or capsules having talc and/or a carbohydrate carried binder or the like are particularly suitable for any oral application. Preferably, carriers for tablets, dragees or capsules include lactose, corn starch and/or potato starch. A syrup or elixir can be used in cases where a sweetened vehicle can be employed.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 500 mg/kg/day.

### EXAMPLES

The novel compounds of the present invention were prepared according to the procedures of the following schemes and examples, using appropriate materials and are further exemplified by the following specific examples. The most preferred compounds of the invention are any or all of those specifically set forth in these examples. These compounds are not, however, to be construed as forming the only genus that is considered as the invention, and any combination of the compounds or their moieties may itself form a genus. The following examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

Preparation 1

### Preparation of (+)-methyl-2-(4-hydroxyphenoxy)-2-methyl butyrate

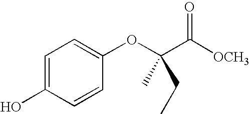

### Step (i): Preparation of 2-[4-phenyl-methoxyphenoxy]-2-methyl butyric acid

To 4-benzyloxyphenol (40 grams, 0.2 mol) was added toluene (400 mL) and sodium hydroxide (powdered) (64 grams, 1.6 mol) at 25-30° C., stirred for 30 minutes and then 2-butanone (180 mL, 2 mol) for 20 minutes was added drop wise. To the reaction mixture was added triethylbenzylammoniumchloride (4 grams) in one portion and the mixture was stirred for 10 minutes. Chloroform (66 mL, 0.8 mol) was added at 25-30° C. very slowly for 30 to 45 minutes by controlling the reaction mixture temperature by cooling intermittently with ice bath, to maintain the temperature below 45° C. The reaction mixture was stirred at 25-30° C. for 13 to 14 hours. The mixture was poured in to water (500 mL) and separated the organic and aqueous layers. The aqueous layer was washed with toluene thrice to remove impurities formed in the reaction. The aqueous layer was acidified (pH to 2) and extracted with ethyl acetate. The organic layer was washed with water, brine and dried over sodium sulphate, and evaporated to get a solid, which was titrated with n-hexane to get 2-[4-phenyl-methoxyphenoxy]-2-methyl butyric acid as a pale yellow solid. (18.8 grams, 32%).

Melting Point: 50-55° C.

¹H NMR (CDCl₃, 400 MHz): δ 7.43-7.30 (m, 5H), 6.92 (d, J=9.13 Hz, 2H), 6.88 (d, J=9.13 Hz, 2H), 5.02 (s, 2H), 2.00-1.83 (m, 2H), 1.42 (s, 3H), 1.05 (t, J=7.53 Hz, 3H).

Mass (m/z): 301 (M+1).

### Step (ii): Preparation of (+)-2-[4-phenyl-methoxyphenoxy]-2-methyl butyric acid

2-[4-Phenyl-methoxyphenoxy]-2-methyl butyric acid (18.8 grams, 62.67 mmol), obtained in step (i), was converted to its R(−)phenylglycinol salt by stirring with the R(−)phenylglycinol (7.7 grams, 56.4 mmol) in ethyl acetate (100 mL) at 25-30° C. for 5 hours to get a suspension of white solid, which was filtered and dried under vacuum to get a racemic acid salt. The resultant salt was recrystallised in ethyl acetate for 3 to 4 times to get a chirally pure (+)enantiomeric salt of 2-[4-phenyl-methoxyphenoxy]-2-methyl butyric acid. The salt was released by treating it with 25% sulphuric acid and extracting it in to ethyl acetate. The ethyl acetate layer was washed with water and dried over sodiumsulphate to give pure (+)-2-[4-phenyl-methoxyphenoxy]-2-methyl butyric acid as white solid (5.2 grams, 43%).

Melting Point: 80-82° C.

¹H NMR (CDCl₃, 400 MHz): δ 7.42-7.30 (m, 5H), 6.91 (d, J=9.13 Hz, 2H), 6.88 (d, J=9.13 Hz, 2H), 5.02 (s, 2H), 2.03-1.82 (m, 2H), 1.42 (s, 3H), 1.05 (t, J=7.52 Hz, 3H).

Mass (m/z): 300 (M⁺).

### Step (iii): Preparation of (+)-methyl-2-[4-phenyl-methoxyphenoxy]-2-methyl butyrate

To the (+)-2-[4-phenyl-methoxyphenoxy]-2-methyl butyric acid (5.2 grams, 17.35 mmol), obtained in step (ii), was added methanol (50 mL) and a catalytic amount of concentrated sulphuric acid (1 mL) and refluxed for 14 hours. The reaction mixture was cooled to 25-30° C. and the methanol was evaporated and the residue was dissolved in ethyl acetate, washed with 10% sodiumbicarbonate solution, water and dried over sodiumsulphate. The solvent was evaporated to give oily (+)-methyl-2-[4-phenyl-methoxyphenoxy]-2-methyl butyrate (5.0 grams, 92%).

¹H NMR (CDCl₃, 200 MHz): δ 7.42-7.30 (m, 5H), 6.85 (d, J=9.41 Hz, 2H), 6.81 (d, J=9.41 Hz, 2H), 4.99 (s, 2H), 3.76 (s, 3H), 1.95-1.92 (m, 2H), 1.42 (s, 3H), 0.97 (t, J=7.53 Hz, 3H).

Mass (m/z): 314 (M⁺).

### Step (iv): Preparation of (+)-methyl-2-[4-hydroxy phenoxy]-2-methyl butyrate

(+)-Methyl-2-[4-phenyl-methoxyphenoxy]-2-methyl butyrate (5.0 grams, 15.92 mmol), obtained in step (iii), was dissolved in methanol (50 mL) and 10% palladium/charcoal (5.0 grams) and ammonium formate (2.0 grams, 31.75 mmol) were added. The mixture was refluxed under nitrogen atmosphere for 45 minutes. The resultant mixture was cooled to 25-30° C. and filtered through celite, washed with methanol and evaporated the solvent to give (+)-methyl-2-[4-hydroxy phenoxy]-2-methyl butyrate as thick oil (3.16 grams, 90%).

¹H NMR (CDCl₃, 200 MHz): δ 6.77 (d, J=8.87 Hz, 2H), 6.69 (d, J=8.87 Hz, 2H), 3.77 (s, 3H), 2.09-1.88 (m, 2H), 1.41 (s, 3H), 0.97 (t, J=7.52 Hz, 3H).

### Preparation 2

### Preparation of (−) methyl-2-(4-hydroxyphenoxy)-2-methyl butyrate

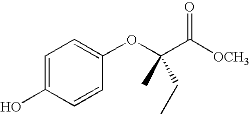

### Step (i): Preparation of 2-[4-phenyl-methoxyphenoxy]-2-methyl butyric acid

To the 4-benzyloxyphenol (40 grams, 0.2 mol) was added toluene (400 mL) and sodium hydroxide (powdered) (64 grams, 1.6 mol) at 25-30° C., stirred for 30 minutes and then 2-butanone (180 mL, 2 mol) for 20 minutes was added drop wise. Triethylbenzyl ammoniumchloride (4 grams) was added in one portion and the mixture was stirred for 10 minutes. Chloroform (66 mL, 0.8 mol) was added at 25-30° C. very slowly for 30 to 45 minutes by controlling the reaction mixture temperature by cooling intermittently with ice bath, to maintain the temperature below 45° C. Then the reaction mixture was stirred at 25-30° C. for 13 to 14 hours. The mixture was poured in to water (500 mL) and separated the organic and aqueous layers. The aqueous layer was washed with toluene thrice to remove impurities formed in the reaction. The aqueous layer was acidified (pH to 2) and extracted with ethyl acetate. The organic layer was washed with water, brine and dried over sodiumsulphate, and evaporated to get a solid, which was titrated with n-hexane to get a reasonably pure 2-[4-phenyl-methoxyphenoxy]-2-methyl butyric acid as a pale yellow solid. (18.8 grams, 32%).

Melting Point: 50-55° C.

¹H NMR (CDCl₃, 400 MHz): δ 7.43-7.30 (m, 5H), 6.92 (d, J=9.13 Hz, 2H), 6.88 (d, J=9.13 Hz, 2H), 5.02 (s, 2H), 2.00-1.83 (m, 2H), 1.42 (s, 3H), 1.05 (t, J=7.53 Hz, 3H).

Mass (m/z): 301 (M+1).

### Step (ii): Preparation of (−)-2-[4-phenyl-methoxyphenoxy]-2-methyl butyric acid

2-[4-Phenyl-methoxyphenoxy]-2-methyl butyric acid (18.8 grams, 62.67 mmol), obtained in step (i), was converted to its S(+)phenylglycenol salt by stirring with the S(+)phenylglycenol (7.7 grams, 56.4 mmol) in ethyl acetate (100 mL) at 25-30° C. for 5 hours to get a suspension of white solid, which was filtered and dried under vacuum to get a salt. This salt of racemic acid was recrystallised in ethyl acetate for 3 to 4 times to get a chirally pure (−)enantiomeric salt of 2-[4-phenyl-methoxyphenoxy]-2-methyl butyric acid. The resultant salt was released by treating it with 25% sulphuric acid and extracting it in to ethyl acetate. The ethyl acetate layer was washed with water and dried over sodiumsulphate to give (−)-2-[4-phenyl-methoxyphenoxy]-2-methyl butyric acid as a pure white solid. (5.2 grams, 43%).

Melting Point: 80-82° C.

¹H NMR (CDCl₃, 400 MHz): δ 7.42-7.30 (m, 5H), 6.91 (d, J=9.13 Hz, 2H), 6.88 (d, J=9.13 Hz, 2H), 5.02 (s, 2H), 2.03-1.82 (m, 2H), 1.42 (s, 3H), 1.05 (t, J=7.52 Hz, 3H).

Mass (m/z): 300 (M⁺).

### Step (iii): Preparation of (−)-methyl-2-[4-phenyl-methoxyphenoxy]-2-methyl butyric acid

To the (−)-2-[4-phenyl-methoxyphenoxy]-2-methyl butyric acid (5.2 grams, 17.35 mmol), obtained in step (ii), was added methanol (50 mL) and catalytic amount of concentrated sulphuric acid (1 mL) and refluxed for 14 hours. The reaction was cooled to 25-30° C. and the methanol was evaporated. The residue was dissolved in ethylacetate, washed with 10% sodium bicarbonate solution, water and dried over sodiumsulphate, evaporated the solvent to give oily (−)-methyl-2-[4-phenyl-methoxyphenoxy]-2-methyl butyrate (5.0 grams, 92%).

¹H NMR (CDCl₃, 200 MHz): δ 7.42-7.30 (m, 5H), 6.85 (d, J=9.41 Hz, 2H), 6.81 (d, J=9.41 Hz, 2H), 4.99 (s, 2H), 3.76 (s, 3H), 1.95-1.92 (m, 2H), 1.42 (s, 3H), 0.97 (t, J=7.53 Hz, 3H).

Mass (m/z): 314 (M⁺).

### Step (iv): Preparation of (−)-methyl-2-[4-hydroxy phenoxy]-2-methyl butyrate

(−)-Methyl-2-[4-phenyl-methoxyphenoxy]-2-methyl butyrate (5.0 grams, 15.92 mmol), obtained in step (iii), was dissolved in ethanol (50 mL). Ten % palladium/charcoal (5.0 grams) and ammoniumformate (2.0 grams, 31.75 mmol) were added and refluxed under nitrogen atmosphere for 45 minutes. The resultant mixture was cooled to 25-30° C. and filtered through celite, washed with methanol and evaporated the solvent to give (−)-methyl-2-[4-hydroxy phenoxy]-2-methyl butyrate as thick oil (3.16 grams, 90%).

¹H NMR (CDCl₃, 200 MHz): δ 6.77 (d, J=8.87 Hz, 2H), 6.69 (d, J=8.87 Hz, 2H), 3.77 (s, 3H), 2.09-1.88 (m, 2H), 1.41 (s, 3H), 0.97 (t, J=7.52 Hz, 3H).

### Example 1

### Preparation of Ethyl-2-{4-[3-(4-benzyloxyphenyl)isoxazol-5-ylmethoxy]phenoxy-2-methyl butyrate

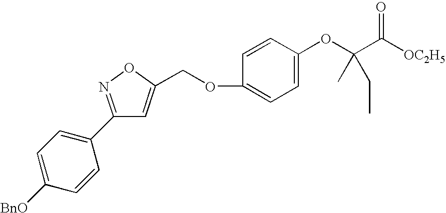 Step (i):

### Preparation of methane sulfonicacid [3-(4-benzyloxy-phenyl)-isoxazol-5-yl methyl]ester

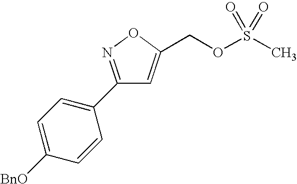

To 3-(4-benzyloxyphenyl)isoxazol-5-yl methanol (Prepared as described in Synthesis, 2002, 12, 1663) (1 grams, 3.56 mmol), was added dichloromethane (20 mL) and triethylamine (1.25 mL, 8.9 mmol). The reaction mixture was cooled to 0° C. and methanesulfonylchloride (0.42 mL, 5.34 mmol) was added drop wise. The resulting mixture was stirred at 25-30° C. for 1 hour followed by diluting with 150 mL dichloromethane and washed with excess of water, brine, and dried over sodiumsulphate. Evaporation of the solvent gives the title compound as a light yellow solid. (1.13 grams, 89%).

Melting Point: 110-112° C.

¹H NMR (CDCl₃, 200 MHz): δ 7.73 (d, J=8.70 Hz, 2H), 7.42-7.30 (m, 5H), 7.05 (d, J=8.70 Hz, 2H), 6.69 (s, 1H), 5.33 (s, 2H), 5.12 (s, 2H), 3.06 (s, 3H).

Mass (m/z): 360 (M+1)

Step (ii):

### Preparation of ethyl-2-{4-[3-(4-benzyloxyphenyl)isoxazol-5-ylmethoxy]phenoxy-2-methyl butyrate

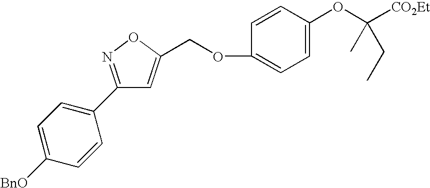

To the ethyl-2-(4-hydroxyphenoxy)-2-methyl butyrate (1.06 grams, 2.94 mmol) was added acetonitrile (20 mL) and anhydrous potassium carbonate (1.2 grams, 8.82 mmol). The mixture was stirred at 70° C. for 30 minutes and the mesylate (1.06 grams, 2.94 mmol), obtained from above step (i), was added to the acetonitrile (20 mL). The mixture was stirred under reflux for about 14 hours and cooled to 25-30° C., filtered off the potassium carbonate and washed with more acetonitrile, and evaporated the solvent. The crude product was purified over silica gel column (120-200 mesh) by eluting with 1:4 ethyl acetate and petroleum ether to get a pure compound as a pale yellow solid (1.30 grams, 88%).

Melting Point: 90-92° C.

¹H NMR (CDCl₃, 200 MHz): δ 7.73 (d, J=8.70 Hz, 2H), 7.42-7.25 (m, 5H), 7.04 (d, J=8.70 Hz, 2H), 6.85 (s, 4H), 6.56 (s, 1H), 5.12 (s, 4H), 4.24 (q, J=7.02 Hz, 2H), 2.05-1.95 (m, 2H), 1.43 (s, 3H), 1.27 (t, J=7.02 Hz, 3H), 0.98 (t, J=7.58 Hz, 3H).

Mass (m/z): 502 (M+1).

### Example 2

The compound of Example 2 was prepared by the procedure by following the procedure as described in Example 1.

| | | |
|---|---|---|
| 2 | 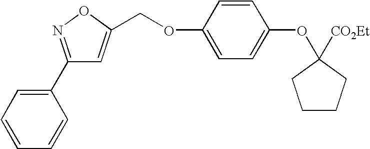 | ¹H NMR (CDCl₃, 400 MHz): δ7.82-7.77(m, 2H), 7.45-7.43(m, 3H), 6.87(d, J = 9.27 Hz, 2H), 6.73(d, J = 9.27 Hz, 2H), 6.62(s, 1H), 5.14(s, 2H), 4.21(q, J = 7.02 Hz, 2H), 2.30-2.10(m, 4H), 1.990-1.70(m, 4H), 1.19(t, J = 7.02 Hz, 3H). MS: 408 (M + 1). Melting point: 83-85° C. |

### Example 3

### Preparation of 2-{4-[3-(4-benzyloxyphenyl)isoxazol-5-ylmethoxy]phenoxy-2-methyl butyric acid

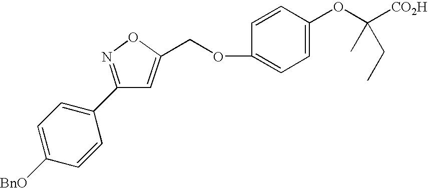

To ethyl-2-{4-[3-(4-benzyloxyphenyl)isoxazol-5-ylmethoxy]phenoxy-2-methyl butyrate (1.29 grams, 2.57 mmol), obtained in Example 1, in 1:1 methanol:tetrahydrofuran (20 mL), was added lithium hydroxide (0.65 grams, 15.45 mmol) dissolved in minimum amount of water at 0° C. The resulting mixture was stirred at 25-30° C. for 14 hours. The solvent was evaporated at 40° C. and the residue was diluted with 50 mL of water and washed with diethyl ether. The aqueous layer was acidified (pH to 2) with 2N hydrochloric acid, extracted with ethyl acetate. The ethyl acetate layer was washed with water, brine, dried over sodium sulphate and the solvent was evaporated to give a white solid (1.0 gram, 83%).

Melting Point: 132-134° C.

¹H NMR (CDCl₃, 200 MHz): δ 7.30 (d, J=8.43 Hz, 2H), 7.41-7.25 (m, 5H), 7.03 (d, J=8.43 Hz, 2H), 6.91 (s, 4H), 6.57 (s, 1H), 5.14 (s, 2H), 5.11 (s, 2H), 2.04 (m, 2H), 1.43 (s, 3H), 1.05 (t, J=7.58 Hz, 3H).

Mass (m/z): 474 (M+1).

### Example 4

### Preparation of 2-{4-[3-(4-hydroxyphenyl)isoxazol-5-ylmethoxy]phenoxy-2-methyl butyric acid

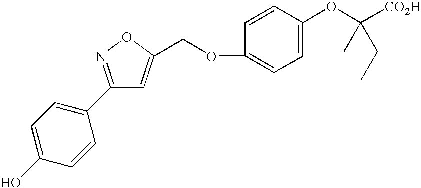

2-{4-[3-(4-Benzyloxyphenyl)isoxazol-5-ylmethoxy]phenoxy-2-methyl butyric acid (1.0 gram, 2.11 mmol), obtained in example 3, was hydrogenated over 10% palladium/charcoal (0.3 gram) in methanol and dioxane mixture (1:1) (30 mL) at 25-30° C. for a period of 2 hours. The mixture was filtered over celite and the solvent evaporated to get a white solid (0.6 grams, 75%).

Melting Point: 142-144° C.

¹H NMR (CDCl₃+DMSO-d⁶, 200 MHz): δ 12.9 (bs, 1H, D₂O exchangeable), 9.9 (bs, 1H, D₂O exchangeable), 7.66 (d, J=8.42, 2H), 6.96-6.83 (m, 7H), 5.19 (s, 2H), 1.90-1.80 (m, 2H), 1.35 (s, 3H), 0.93 (t, J=7.30 Hz, 3H).

Mass (m/z): 384 (M+1).

### Example 5

### Preparation of 2-{4-[3-(4-methanesulfonyloxyphenyl)isoxazol-5-ylmethoxy]phenoxy-2-methyl butyric acid

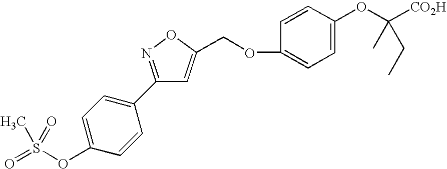

To 2-{4-[3-(4-hydroxyphenyl)isoxazol-5-ylmethoxy]phenoxy-2-methyl butyric acid (0.6 grams, 1.56 mmol), obtained in example 4, was added dichloromethane (10 mL) and triethylamine (0.66 mL, 4.69 mmol). The mixture was cooled to 0° C., and methanesulfonylchloride (0.37 mL, 3.92 mmol) was added to the above mixture and the reaction mixture was stirred at 25-30° C. for 1 hour. The mixture was diluted with 100 mL more of dichloromethane and washed with water, brine, dried over sodiumsulphate and evaporated to dryness to get a brown mass, which was dissolved in 5 mL of tetrahydrofuran. Saturated sodium bicarbonate solution was added until the mixture is alkaline (pH at 8) and the mixture was stirred for 1 hour. The tetrahydrofuran was evaporated and the aqueous solution was acidified (pH to 2), extracted with ethyl acetate, washed with water, brine, dried over sodiumsulphate and evaporated to remove the solvent. The crude product was purified with silica gel column chromatography by eluting with 1% methanol in chloroform to give thick gummy syrup (0.4 gram, 55%).

¹H NMR (CDCl₃, 200 MHz): δ 7.87 (d, J=8.54 Hz, 2H), 7.39 (d, J=8.54 Hz, 2H), 6.95 (d, J=9.15 Hz, 2H), 6.90 (d, J=9.15 Hz, 2H), 6.64 (s, 1H), 5.18 (s, 2H), 3.19 (s, 3H), 2.00-1.87 (m, 2H), 1.43 (s, 3H), 1.05 (t, J=7.32 Hz, 3H).

Mass (m/z): 462 (M+1).

### Examples 6 and 7

The compounds of Examples 6 and 7 were prepared by following the procedure as described in Example 5, by taking appropriate chiral starting materials

| | | |
|---|---|---|
| 6 | 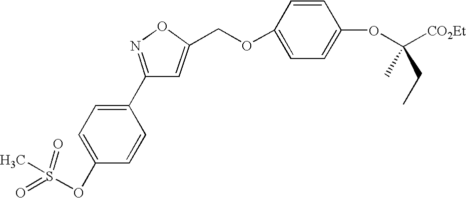 | ¹H NMR: (CDCl₃, 200 MHz): δ7.87(d, J = 8.54 Hz, 2H), 7.39(d, J = 8.54 Hz, 2H), 6.95(d, J = 9.15 Hz, 2H), 6.90(d, J = 9.15 Hz, 2H), 6.64(s, 1H), 5.18(s, 2H), 3.19(s, 3H), 2.00- 1.87(m, 2H), 1.43(s, 3H), 1.05 (t, J = 7.32 Hz, 3H). Mass (m/z): 462 (M + 1). [α]²⁵ = 10.6 (1%, MeOH). |
| 7 | 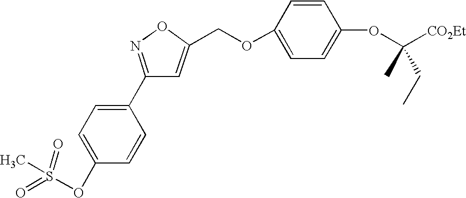 | ¹H NMR: (CDCl₃, 200 MHz): δ7.87(d, J = 8.54 Hz, 2H), 7.39(d, J = 8.54 Hz, 2H), 6.95(d, J = 9.15 Hz, 2H), 6.90(d, J = 9.15 Hz, 2H), 6.64(s, 1H), 5.18(s, 2H), 3.19(s, 3H), 2.00- 1.87(m, 2H), 1.43(s, 3H), 1.05 (t, J = 7.32 Hz, 3H). Mass (m/z): 462 (M + 1). [α]²⁵ =−3.0 (0.1%, MeOH). |

### Examples 8-24

The compounds of Examples 8-24 were prepared by following the procedure as described in Examples 3 and 4, by taking appropriate starting materials.

| | | |
|---|---|---|
| 8 | 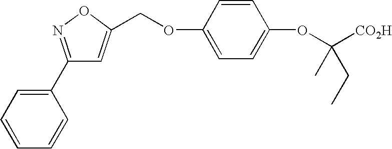 | ¹H NMR (CDCl₃, 400 MHz): δ 7.85- 7.80(m, 2H), 7.50-7.43(m, 3H), 6.95(d, J = 9.40 Hz, 2H), 6.91(d, J = 9.40 Hz, 2H), 6.65(s, 1H), 5.17(s, 2H), 2.01-1.85(m, 2H), 1.43(s, 3H), 1.05(t, J = 7.25 Hz, 3H). Mass (m/z): 368 (M + 1). Melting Point: 70-74° C. |
| 9 | 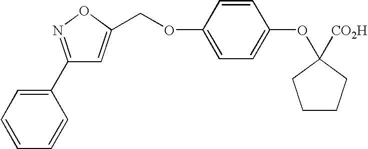 | ¹H NMR (CDCl₃, 400 MHz): δ 7.80- 7.78(m, 2H), 7.46-7.42(m, 3H), 6.89(d, J = 9.13 Hz, 2H), 6.80(d, J = 9.13 Hz, 2H), 6.62(s, 1H), 5.14(s, 2H), 2.28-2.15(m, 4H), 1.84-1.79(m, 4H).]Mass (m/z): 380 (M + 1). Melting Point: 135-137° C. |
| 10 | 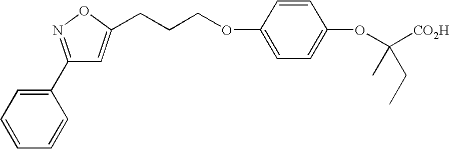 | ¹H NMR (CDCl₃, 400 MHz): δ 7.79- 7.76(m, 2H), 7.45-7.43(m, 3H), 6.91(d, J = 8.86 Hz, 2H), 6.81(d, J = 8.86 Hz, 2H), 6.33(s, 1H) 4.01(t, J = 5.91 Hz, 2H), 3.02(t, J = 7.53 Hz, 2H), 2.25-2.21(m, J = 2H), 1.97-1.83(m, 2H), 1.41(s, 3H), 1.05(t, J = 7.26 Hz, 3H). Mass (m/z): 396 (M + 1). Melting Point: 115-117° C. |
| 11 | 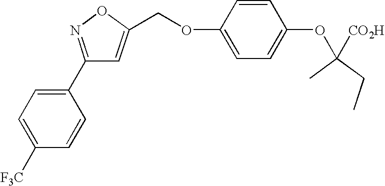 | ¹H NMR (CDCl₃, 400 MHz): δ 7.93(d, J = 8.06 Hz, 2H), 7.72(d, J = 8.06 Hz, 2H), 6.96(d, J = 9.40 Hz, 2H), 6.92(d, J = 9.40 Hz, 2H), 6.68(s, 1H), 5.19(s, 2H), 2.01-1.84(m, 2H), 1.43(s, 3H), 1.05(t, J = 7.52 Hz, 3H). Mass (m/z): 436 (M + 1). Melting Point: 136-138° C. |
| 12 | 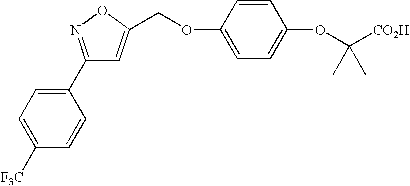 | ¹H NMR (CDCl₃, 400 MHz): δ 7.93(d, J = 8.33 Hz, 2H), 7.72(d, J = 8.33 Hz, 2H), 6.93(d, J = 9.40 Hz, 2H), 6.91(d, J = 9.40 Hz, 2H), 6.68(s, 1H), 5.19(s, 2H), 1.55(s, 6H). Mass (m/z): 422 (M + 1) Melting Point: 166-168° C. |
| 13 | 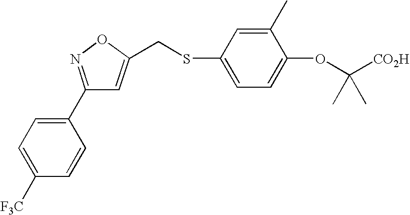 | ¹H NMR (CDCl₃, 400 MHz): δ 7.85(d, J = 8.06 Hz, 2H), 7.68(d, J = 8.06 Hz, 2H), 7.22(s, 1H), 7.14(d, J = 8.60 Hz, 1H), 6.71(d, J = 8.60 Hz, 1H), 6.34(s, 1H), 4.10(s, 2H), 2.19(s, 3H), 1.61(s, 6H). Mass (m/z): 452 (M + 1). Melting Point: 106-108° C. |
| 14 | 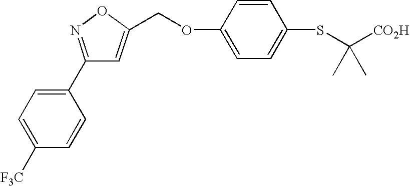 | ¹H NMR (CDCl₃, 400 MHz): δ 7.91(d, J = 8.06 Hz, 2H), 7.71(d, J = 8.06 Hz, 2H), 7.48(d, 8.87 Hz, 2H), 6.95(d, J = 8.87 Hz, 2H), 6.68(s, 1H), 5.21(s, 2H), 1.48(s, 6H). Mass (m/z): 438 (M + 1). Melting Point: 120-122° C. |
| 15 | 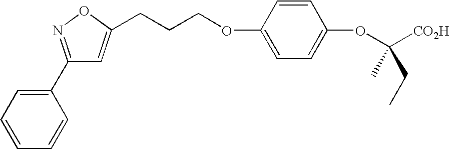 | ¹H NMR (CDCl₃, 400 MHz): δ 7.79-7.76(m, 2H), 7.45-7.43(m, 3H), 6.91(d, J = 8.86 Hz, 2H), 6.81(d, J = 8.86 Hz, 2H), 6.33(s, 1H) 4.01(t, J = 5.91 Hz, 2H), 3.02(t, J = 7.53 Hz, 2H), 2.25-2.21(m, 2H), 1.97-1.83(m, 2H), 1.41(s, 3H), 1.05(t, J = 7.26 Hz, 3H). Mass (m/z): 396 (M + 1). Melting Point: 105-108° C. |
| 16 | 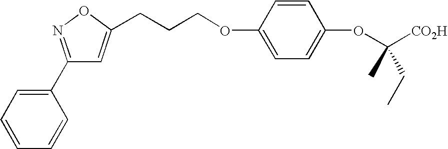 | ¹H NMR (CDCl₃, 400 MHz): δ 7.79- 7.76(m, 2H), 7.45-7.43(m, 3H), 6.91(d, J = 8.86 Hz, 2H), 6.81(d, J = 8.86 Hz, 2H), 6.33(s, 1H) 4.01(t, 1 = 5.91 Hz, 2H), 3.02(t, J = 7.53 Hz, 2H), 2.25-2.21(m, 2H), 1.97-1.83(m, 2H), 1.41(s, 3H), 1.05(t, J = 7.26 Hz, 3H). Mass (m/z): 396 (M + 1). Melting Point: 121-122° C. |
| 17 | 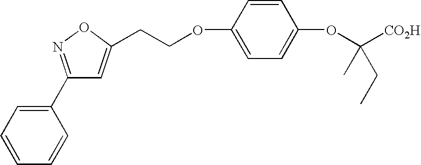 | ¹H NMR (CDCl₃, 400 MHz): δ 7.91(d, J = 8.06 Hz, 2H), 7.01(d, J = 8.06 Hz, 2H), 6.92(d, J = 8.87 Hz, 2H), 6.87-6.74(m, 3H), 6.50(s, 1H), 4.30(t, J = 6.14 Hz, 2H), 3.30(t, J = 6.14 Hz, 2H), 2.00-1.83(m, 2H), 1.42(s, 3H), 1.05(t, 7.26 Hz, 3H). Mass (m/z): 449 (M + 1). |
| 18 | 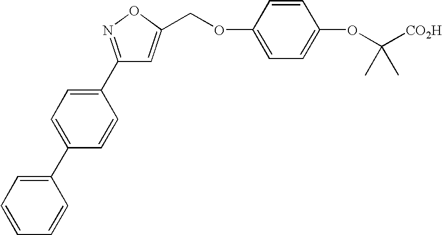 | ¹H NMR (d⁶DMSO, 400 MHz): δ 7.97(d, J = 8.33 Hz, 2H), 7.82(d, J = 8.33 Hz, 2H), 7.74(d, J = 8.60 Hz, 2H), 7.52-7.44(m, 3H), 7.22(s, 1H), 6.99(d, J = 9.14 Hz, 2H), 6.85(d, J = 9.14 Hz, 2H), 5.27(s, 2H), 1.50(s, 6H). Mass (m/z): 430 (M + 1). Melting Point: 168-170° C. |
| 19 | 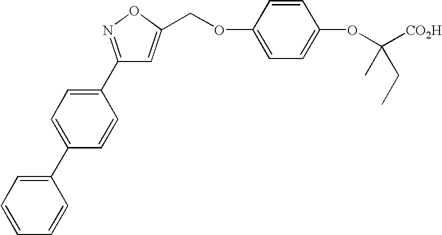 | ¹H NMR (d⁶DMSO, 400 MHz): δ 7.97(d, J = 8.32 Hz, 2H), 7.72(d, J = 8.60 Hz, 2H), 7.82(d, J = 8.32 Hz, 2H), 7.75-7.39(m, 3H), 7.20(s, 1H), 6.90(d, J = 9.14 Hz, 2H), 6.85(d, 9.14 Hz, 2H), 5.27(s, 2H), 1.78-1.35(m, 2H), 1.27(s, 3H), 0.85(t, J = 7.52 Hz, 3H). Mass (m/z): 444 (M + 1). Melting Point: 188-190° C. |
| 20 | 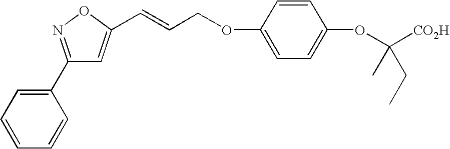 | ¹H NMR (CDCl₃, 400 MHz): δ 7.81- 7.77(m, 2H), 7.47-7.42(m, 3H), 7.25(s, 1H), 6.95(d, J = 7.52 Hz, 2H), 6.88(d, J = 7.52 Hz, 2H), 6.73(s, 1H), 6.50(s, 1H), 4.73(d, J = 2.15 Hz, 2H), 2.10-1.88(m, 2H), 1.43(s, 3H), 1.06(t, J = 7.25 Hz, 3H). Mass (m/z): 394 (M + 1). Melting Point: 118-120° C. |
| 21 | 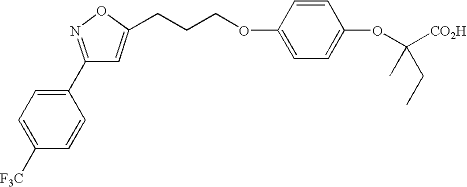 | ¹H NMR (CDCl₃, 400 MHz): δ 7.90(d, J = 8.06 Hz, 2H), 7.70(d, J = 8.06 Hz, 2H), 6.91(d, J = 9.13 Hz, 2H), 6.81(d, J = 9.13 Hz, 2H), 6.37(s, 1H), 4.01(t, J = 5.91 Hz, 2H), 3.05(t, J = 7.52 Hz, 2H), 2.27-2.22(m, 2H), 2.00-1.84(m, 2H), 1.42(s, 3H), 1.05(t, J = 7.52 Hz, 3H). Mass (m/z): 463 (M+) Melting Point: 110-112° C. |
| 22 | 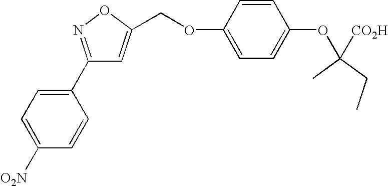 | ¹H NMR (CDCl₃, 400 MHz): δ 8.32(d, J = 8.86 Hz, 2H), 7.99(d, J = 8.86 Hz, 2H), 6.96(d, J = 9.13 Hz, 2H), 6.91(d, J = 9.13 Hz, 2H), 6.72(s, 1H), 5.20(s, 2H), 2.00-1.88(m, 2H), 1.44(s, 3H), 1.05(t, J = 7.52 Hz, 3H). Mass (m/z): 412 (M⁺). Melting Point: 128-130° C. |
| 23 | 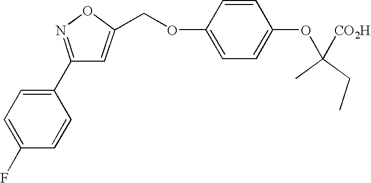 | ¹H NMR (CDCl₃, 400 MHz): δ 7.80- 7.76(m, 2H) 7.16-7.11(m, 2H), 6.95(d, J = 9.14 Hz, 2H), 6.90(d, J = 9.14 Hz, 2H), 6.60(s, 1H), 5.16(s, 2H), 2.00-1.84(m, 2H), 1.43(t, 3H), 1.05(t, 7.52 Hz, 3H). Mass (m/z): 386 (M + 1). Melting Point: 118-120° C. |
| 24 | 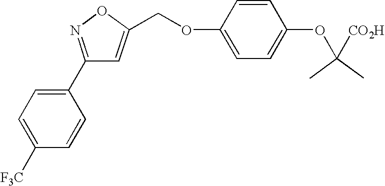 | ¹H NMR (CDCl₃ + d⁶DMSO, 400 MHz): δ7.93(d, J = 8.05 Hz, 2H), 7.10(d, J = 8.05 Hz, 2H), 6.81(d, J = 8.87 Hz, 2H), 6.63(s, 1H), 6.57(d, J = 8.87 Hz, 2H), 5.10(bs, 1 H, D₂O exchangeable), 4.47(s, 2H), 1.47(s, 6H). Mass (m/z): 420 (M + 1). Melting Point: 120-121° C. |

### Example 25

### Preparation of L-Arginine salt of 2-{4-[3-(4-methanesulfonyloxyphenyl)isoxazol-5-ylmethoxy]phenoxy-2-methyl butyric acid

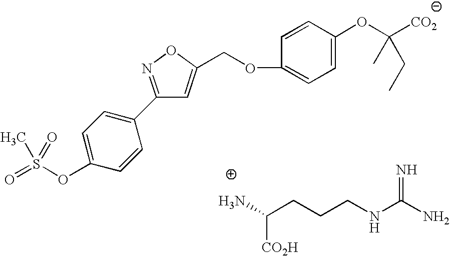

To 2-{4-[3-(4-Methanesulfonyloxyphenyl)isoxazol-5-ylmethoxy]phenoxy-2-methyl butyric acid (0.4 gram, 0.868 mmol), obtained in example 5, taken in dry methanol (4 mL), was added L-arginine (0.136 gram, 0.781 mmol) at 25-30° C., and the mixture stirred at 25° C. for 14 hours. The solvent was evaporated under reduced pressure, and the residue titrated with diethyl ether and dried under vacuum for 8 hours to get a white solid (0.5 gram, 91%).

Melting Point: 112-114° C.

¹H NMR (DMSO-d⁶, 200 MHz): δ 7.80 (d, J=8.86 Hz, 2H), 7.49 (d, J=8.86 Hz, 2H), 7.19 (s, 1H), 6.89 (d, J=9.14 Hz, 2H), 6.82 (d, J=9.14 Hz, 2H), 5.22 (s, 2H), 3.40-3.30 (m, 1H), 3.17 (s, 3H), 3.07-3.01 (m, 2H), 2.53-2.40 (m, 2H), 177-1.70 (m, 2H), 1.62-1.50 (m, 2H), 1.26 (s, 3H), 0.84 (t, J=7.52 Hz, 3H).

Mass (m/z): 636 (M+1).

### Examples 26 and 27

The compounds of Examples 26 and 27 were prepared by following the procedure as described in Example 25, by taking appropriate starting materials.

| | | |
|---|---|---|
| 26 | 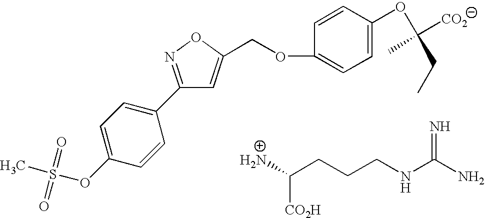 | ¹H NMR (DMSO-d⁶, 400 MHz): δ 8.00(d, J = 8.60 Hz, 2H), 7.49(d, J = 8.60 Hz, 2H), 7.19(s, 1H), 6.89(d, J = 9.14 Hz, 2H), 6.83(d, J = 9.14 Hz, 2H), 5.23(s, 2H), 3.40- 3.20(m, 3H), 3.17(s, 3H), 3.11-3.00(m, 2H), 1.81-1.61(m, 2H), 1.60-1.55(m, 2H), 1.27(s, 3H), 0.84(t, J = 7.26 Hz, 3H). Mass (m/z): 636 (M + 1). Melting Point: 94-96° C. |
| 27 | 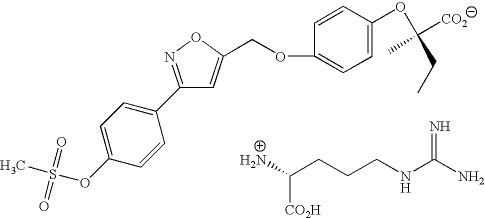 | ¹H NMR (DMSO-d⁶, 400 MHz): δ 8.00(d, J = 8.60 Hz, 2H), 7.49(d, J = 8.60 Hz, 2H), 7.19(s, 1H), 6.89(d, J = 9.14 Hz, 2H), 6.83(d, J = 9.14 Hz, 2H), 5.23(s, 2H), 3.40- 3.20(m, 3H), 3.17(s, 3H), 3.11-3.00(m, 2H), 1.81-1.61(m, 2H), 1.60-1.55(m, 2H), 1.27(s, 3H), 0.84(t, J = 7.26 Hz, 3H). Mass (m/z): 636 (M + 1) Melting Point: 110-114° C. |

### Example 28

### Preparation of 2-methyl-2-{4-[3-(3-phenylisoxazol-5-yl)propyl]phenoxy}propanoic acid

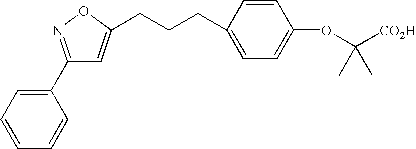 Step (i):

### Preparation of 5-[3-(4-methoxyphenyl)propyl]-3-phenylisoxazole

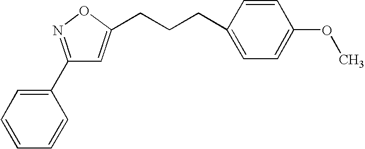

To benzohydroximinoyl chloride (Reference: J. Org. Chem., 1980, 45, 3916) (0.223 grams, 2.87 mmol) in chloroform was added 1-methoxy-4-pent-4-ynyl benzene (Reference: GB: 975591; Chem. Abstracts, 1965, 62, 7692) (0.5 grams, 2.87 mmol) followed by NEt₃ (0.24 mL, 1.72 mmol) at 0° C. The resultant mixture was stirred at 55 to 60° C. for 3 to 4 hours. The mixture was cooled to 25-30° C., diluted with 100 mL more of chloroform and washed with water and brine. The resultant mixture was dried over sodium sulphate and evaporated the solvent. The resulting crude product was titrated with n-hexane to give pure white solid. Yield: 0.646 gram (77%).

¹H NMR (CDCl₃, 200 MHz): δ 7.81-7.76 (m, 2H), 7.45-7.40 (m, 3H), 7.11 (d, J=8.42 Hz, 2H), 6.84 (d, J=8.42 Hz, 2H), 6.28 (s, 1H), 3.79 (s, 3H), 2.80 (t, J=7.58 Hz, 2H), 2.67 (t, J=7.30 Hz, 2H), 2.12-2.01 (m, 2H).

Mass (m/z): 294 (M+1).

Step (ii):

### Preparation of 5-[3-(4-hydroxyphenyl)propyl]3-phenylisoxazole

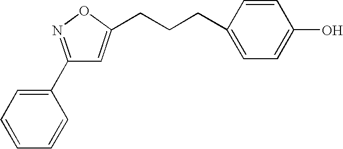

5-[3-(4-methoxyphenyl)propyl]3-phenylisoxazole (0.642 grams, 2.19 mmol), obtained from step (i), in dry dichloromethane (35 mL) was cooled to −78° C. and boronbromide (0.77 mL, 4.6 mmol) was added. The resulting mixture was allowed to reach 25-30° C. and stirred for 2 hours. After completion of the reaction, water was added and extracted with dichloromethane. The organic layer was washed with 5% sodium bicarbonate solution and then with brine, dried over sodiumsulphate and the solvent evaporated to get a pure gummy material (Yield: 0.605 mg, 99%).

¹H NMR (CDCl₃, 200 MHz): δ 7.80-7.75 (m, 2H), 7.44-7.24 (m, 3H), 7.04 (d, J=8.43 Hz, 2H), 6.79 (d, J=8.43 Hz, 2H), 6.29 (s, 1H), 6.23 (bs, 1H, D₂O exchangeable), 2.79 (t, J=7.58 Hz, 2H), 2.64 (t, J=7.30 Hz, 2H), 2.10-1.96 (m, 2H).

Mass (m/z): 280 (M+1).

Step (iii):

### Preparation of ethyl-2-methyl-2-{4-[3-(3-phenylisoxazol-5-yl)propyl]phenoxy}propionate

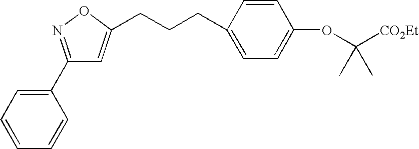

To 5-[3-(4-hydroxyphenyl)propyl]3-phenylisoxazole (0.6 grams, 2.15 mmol), obtained in step (ii), in dimethylformamide (20 mL), was added potassium carbonate (0.89 grams, 6.45 mmol) and stirred at 25-30° C. for 0.5 hour. Ethyl-2-bromoisobutyrate (0.38 mL, 2.58 mmol) was then added, and the resultant mixture stirred at 80° C. for 14 hours. The potassium carbonate was filtered off, and the filtrate diluted with water and extracted with ethyl acetate. The organic layer was washed with water several times, then with brine, dried over sodiumsulphate and the solvent evaporated under reduced pressure to give a crude compound as an oil. The crude compound was purified on silica gel column by eluting with 15% ethyl acetate and petroleum ether to give a pure compound as an oil (Yield: 0.388 mg, 40%).

¹H NMR: (CDCl₃, 200 MHz): δ 7.80-7.77 (m, 2H), 7.44-7.25 (m, 3H), 7.05 (d, J=8.42 Hz, 2H), 6.78 (d, J=8.42 Hz, 2H), 4.23 (q, J=7.02 Hz, 2H), 2.79 (t, J=7.59 Hz, 2H), 2.65 (t, J=7.30 Hz, 2H), 2.11-1.96 (m, 2H), 1.57 (s, 6H), 1.25 (t, J=7.02 Hz, 3H).

Mass (m/z): 394 (M+1).

Step (iv):

### Preparation of 2-methyl-2-{4-[3-(3-phenylisoxazol-5-yl)propyl]phenoxy}propanoic acid

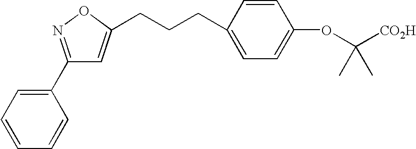

Ethyl-2-methyl-2-{4-[3-(3-phenylisoxazol-5-yl)propyl]phenoxy}propionate (0.338 grams, 0.860 mmol), obtained from step (iii), was hydrolyzed with lithiumhydroxide (0181 grams, 4.3 mmol) as described in step (iii), to get the title compound as a white solid.

Yield: 0.265 gram (84%).

Melting Point: 66-70° C.

¹H NMR (CDCl₃, 400 MHz): δ 7.79-7.69 (m, 2H), 7.45-7.41 (m, 3H), 7.11 (d, J=8.60 Hz, 2H), 6.89 (d, J=8.60 Hz, 2H), 6.28 (s, 1H), 2.80 (t, J=7.26 Hz, 2H), 2.68 (t, J=7.79 Hz, 2H), 2.16-2.02 (m, 2H), 1.58 (s, 6H).

Mass (m/z): 366 (M+1).

### Examples 29-34

The compounds of Examples 29-34 were prepared by procedures similar to those described in Examples 1-28.

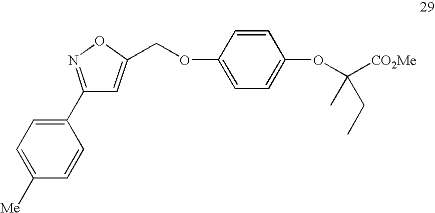

### 2-Methyl-2-[4-(3-(4-methylphenyl)isoxazol-5-ylmethoxy)phenoxy]butyric acid, methyl ester

White solid. Mp: 100-102° C.

¹H-NMR (400 MHz, CDCl₃) δ: 0.97 (t, J=7.5 Hz, 3H); 1.44 (s, 3H); 1.89-1.98 (m, 2H); 2.39 (s, 3H); 3.77 (s, 3H); 5.13 (s, 2H); 6.59 (s, 1H); 6.82-6.88 (aromatics, 4H); 7.26 (d, J=8.0 Hz, 2H); 7.69 (d, J=8.0 Hz, 2H).

Mass (ES) m/z: 396.2 [M+1], 413.4 [M+NH₄⁺], 418 [M+Na⁺], 808.3 [M₂+NH₄⁺], 813.5 [M₂+Na⁺].

IR (KBr) cm⁻¹: 3112, 2939, 1745, 1734.

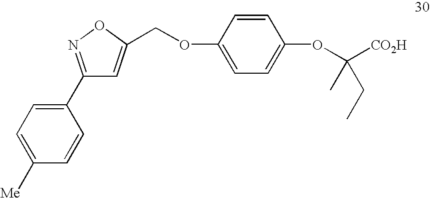

### 2-Methyl-2-[4-(3-(4-methylphenyl)isoxazol-5-ylmethoxy)phenoxy]butyric acid

White solid. Mp: 138-140° C.

¹H-NMR (400 MHz, CDCl₃) δ: 1.05 (t, J=7.25 Hz, 3H); 1.43 (s, 3H); 1.85-1.93 (m, 1H); 1.94-2.01 (m, 1H); 2.40 (s, 3H); 5.16 (s, 2H); 6.61 (s, 1H); 6.89-6.96 (aromatics, 4H); 7.26 (d, J=8.4 Hz, 2H); 7.69 (d, J=8.4 Hz, 2H).

Mass (ES) m/z: 382.3 [M+1], 399.4 [M+NH₄⁺], 404.2 [M+Na⁺].

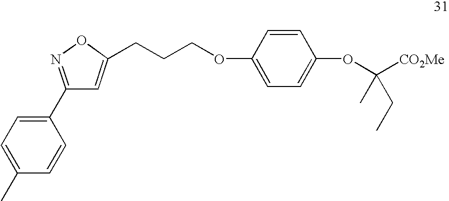

### 2-Methyl-2-{4-[3-(3-(4-methylphenyl)isoxazol-5-yl)propoxy]phenoxy}butyric acid, methyl ester

¹H-NMR (200 MHz, CDCl₃) δ: 0.97 (t, J=7.5 Hz, 3H); 1.42 (s, 3H); 1.88-2.00 (m, 2H); 2.13-2.28 (m, 2H); 2.39 (s, 3H); 3.00 (t, J=7.4 Hz, 2H); 3.77 (s, 3H); 3.98 (t, J=6 Hz, 2H); 6.29 (s, 1H); 6.73-6.85 (aromatics, 4H); 7.24 (d, J=8.2 Hz, 2H); 7.67 (d, J=8.2 Hz, 2H).

Mass (CI) m/z: 424 [M+1].

IR (neat) cm⁻¹: 2953, 1730, 1508.

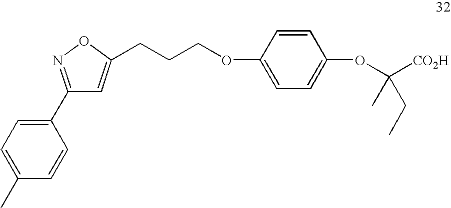

### 2-Methyl-2-{4-[3-(3-(4-methylphenyl)isoxazol-5-yl)propoxy]phenoxy}butyric acid

Cream color solid. Mp: 98-100° C.

¹H-NMR (400 MHz, CDCl₃) δ: 1.05 (t, J=7.3 Hz, 3H); 1.41 (s, 3H); 1.82-1.90 (m, 1H); 1.91-2.00 (m, 1H); 2.20-2.24 (m, 2H); 2.39 (s, 3H); 3.01 (t, J=7.4 Hz, 2H); 4.01 (t, J=6 Hz, 2H); 6.30 (s, 1H); 6.82 (d, J=9.1 Hz, 2H); 6.91 (d, J=9.1 Hz, 2H); 7.25 (d, J=8 Hz, 2H); 7.67 (d, J=8 Hz, 2H).

Mass (CI) m/z: 410 [M+1].

IR (neat) cm⁻¹: 3500, 2923, 1706, 1508, 1214.

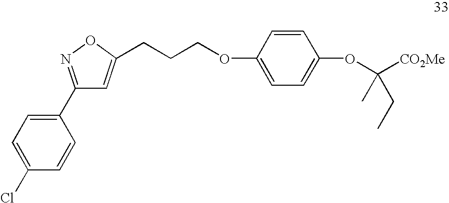

### 2-Methyl-2-{4-[3-(3-(4-chlorophenyl)isoxazol-5-yl)propoxy]phenoxy}butyric acid, methyl ester

¹H-NMR (400 MHz, CDCl₃) δ: 0.97 (t, J=7.5 Hz, 3H); 1.42 (s, 3H); 1.88-1.97 (m, 2H); 2.13-2.24 (m, 2H); 3.01 (t, J=7.5 Hz, 2H); 3.77 (s, 3H); 3.98 (t, J=6.1 Hz, 2H); 6.29 (s, 1H); 6.76 (d, J=9.3 Hz, 2H); 6.82 (d, J=9.3 Hz, 2H); 7.42 (d, J=8.6 Hz, 2H); 7.72 (d, J=8.6 Hz, 2H).

Mass (CI) m/z: 444 [³⁵M+1], 446 [³⁷M+1].

IR (neat) cm⁻¹: 2970, 1738, 1508.

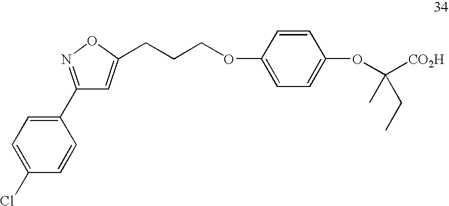

### 2-Methyl-2-{4-[3-(3-(4-chlorophenyl)isoxazol-5-yl)propoxy]phenoxy}butyric acid

Almost white solid. Mp: 130-132° C.

¹H-NMR (400 MHz, CDCl₃) δ: 1.05 (t, J=7.5 Hz, 3H); 1.41 (s, 3H); 1.83-1.91 (m, 1H); 1.92-2.00 (m, 1H); 2.19-2.26 (m, 2H); 3.02 (t, J=7.5 Hz, 2H); 4.01 (t, J=6 Hz, 2H); 6.30 (s, 1H); 6.81 (d, J=9.1 Hz, 2H); 6.92 (d, J=9.1 Hz, 2H); 7.42 (d, J=8.6 Hz, 2H); 7.72 (d, J=8.6 Hz, 2H).

Mass (CI) m/z: 430 [³⁵M+1], 432 [³⁷M+1].

IR (neat) cm⁻¹: 2940, 1712, 1505.

### Examples 35-38

The compounds of Examples 35-38 were prepared by procedures similar to those described in Examples 1-28.

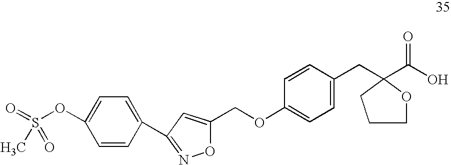

¹H NMR (CDCl₃, 400 MHz): δ 8.00 (d, J=8.6 Hz, 2H), 7.50 (d, J=8.6 Hz, 2H), 7.20 (s, 1H), 7.18 (d, J=8.3 Hz 2H), 6.96 (d, J=8.6 Hz 2H), 5.30 (s, 2H), 3.76 (t, J=6.6 Hz, 2H), 3.43-(s, 3H), 3.04 (d, J=13.7 Hz 1H), 2.85 (d, J=13.4 Hz 1H), 2.14-2.07 (m, 1H), 1.86-1.81 (m, 1H), 1.79-1.71 (m, 1H), 1.69-1.64 (m, 1H).

Mass (m/z): 428 (—COOH).

Melting Point: 106-108° C.

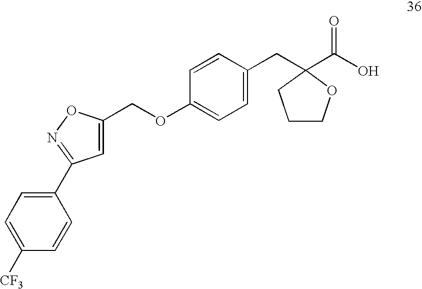

¹H NMR (400 MHz, CDCl₃): δ 7.92 (d, J=8.0 Hz, 2H), 7.72 (d, J=8.0 Hz, 2H), 7.21-7.18 (m, 2H), 6.92-6.89 (m, 2H), 6.67 (s, 1H), 5.19 (s, 2H), 4.13-3.92 (m, 1H), 3.90-3.87 (m, 1H), 3.20 (d, J=7.0 Hz, 1H), 2.90 (d, J=7.0 Hz, 1H), 2.40-2.35 (m, 1H), 2.09-1.90 (m, 1H), 1.89-1.79 (m, 2H).

Mass (m/z): 448 (M+1)⁺, 402, 332, 303, 275, 244, 177, 106.

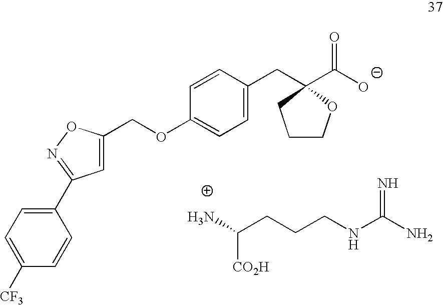

¹H NMR (CDCl₃, 400 MHz): δ 8.05 (d, J=8.3 Hz, 2H), 7.79 (d, J=8.6 Hz, 2H), 7.24 (d, J=8.6 Hz, 2H), 7.00 (s, 2H), 6.91 (d, J=8.6 Hz 2H), 5.25 (s, 2H), 3.87-3.81 (m, 1H), 3.79-3.74-(m, 1H), 3.53 (t, J=6.0 Hz 1H), 3.20-3.11 (m, 4H), 2.89-2.85 (m, 1H), 2.24-2.17 (m, 1H) 1.89-1.74 (m, 4H), 1.69-1.57 (m, 2H), 1.34-1.19 (m, 1H).

Mass (m/z): 448 (−acid).

Melting point: 150-155° C.

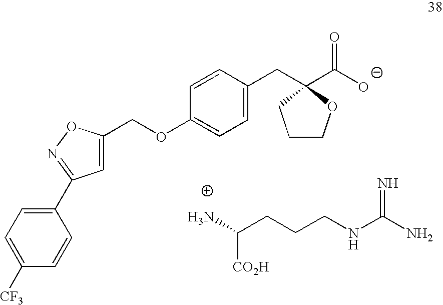

¹H NMR (CDCl₃, 400 MHz): δ 8.05 (d, J=8.0 Hz, 2H), 7.79 (d, J=8.3 Hz, 2H), 7.24 (d, J=8.6 Hz, 2H), 7.00 (s, 2H), 6.96 (d, J=8.6 Hz 2H), 5.25 (s, 2H), 3.85-3.80 (m, 1H), 3.79-3.74 (m, 1H), 3.40 (t, J=5.4 Hz 1H), 3.20-3.11 (m, 4H), 2.88-2.84 (m, 1H), 2.21-2.18 (m, 1H) 1.86-1.81 (m, 4H), 1.68-1.61 (m, 2H), 1.34-1.19 (m, 1H).

Mass (m/z): 448 (−acid)+.

Melting point: 168-170° C.

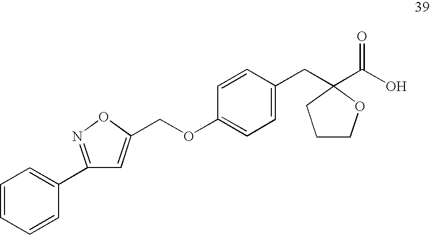

### Examples 40-54

The compounds of Examples 40-54 can be prepared by the person skilled in the art by following any of the procedures described in Examples 1-38.

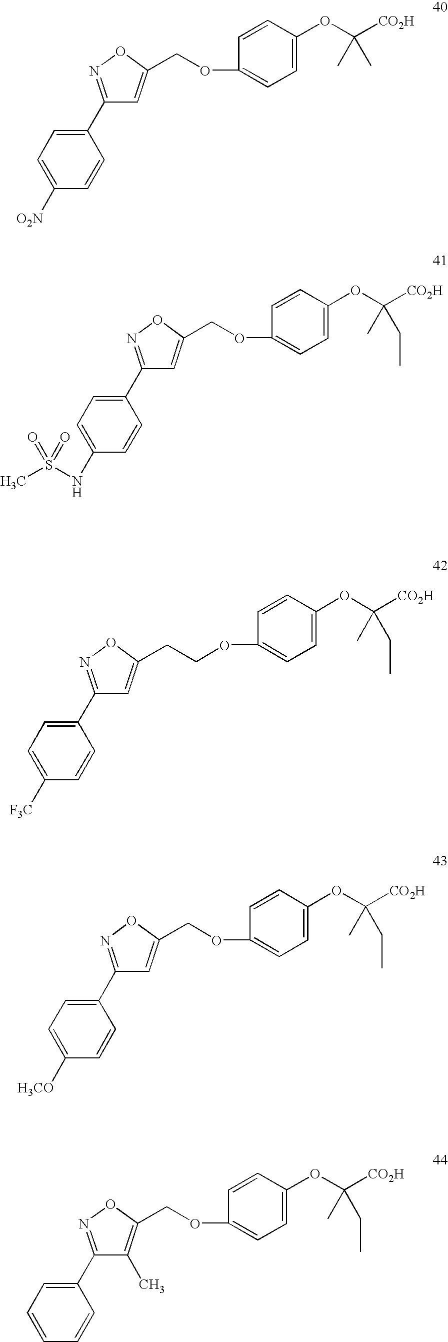

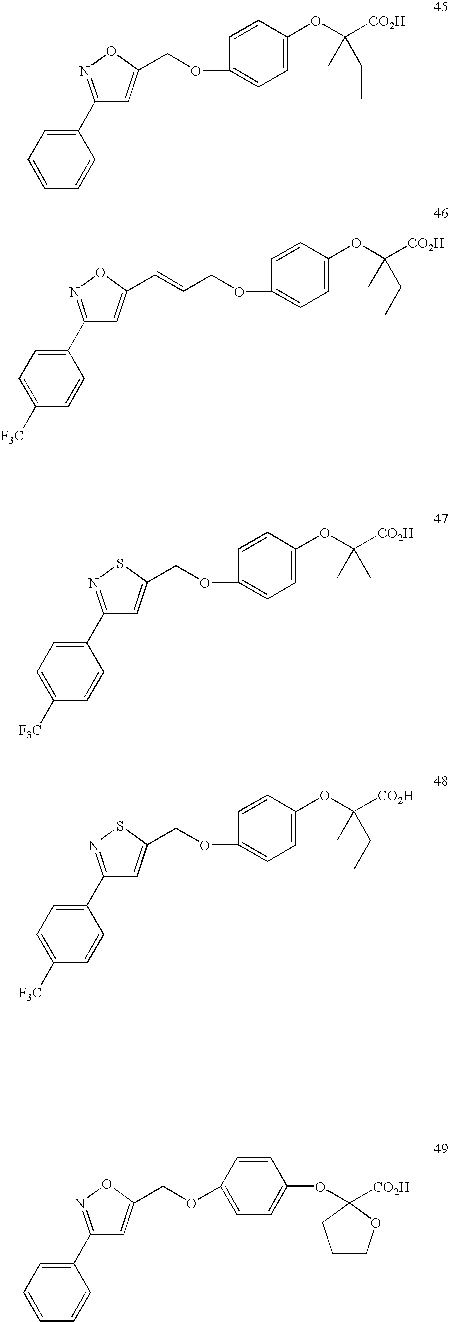

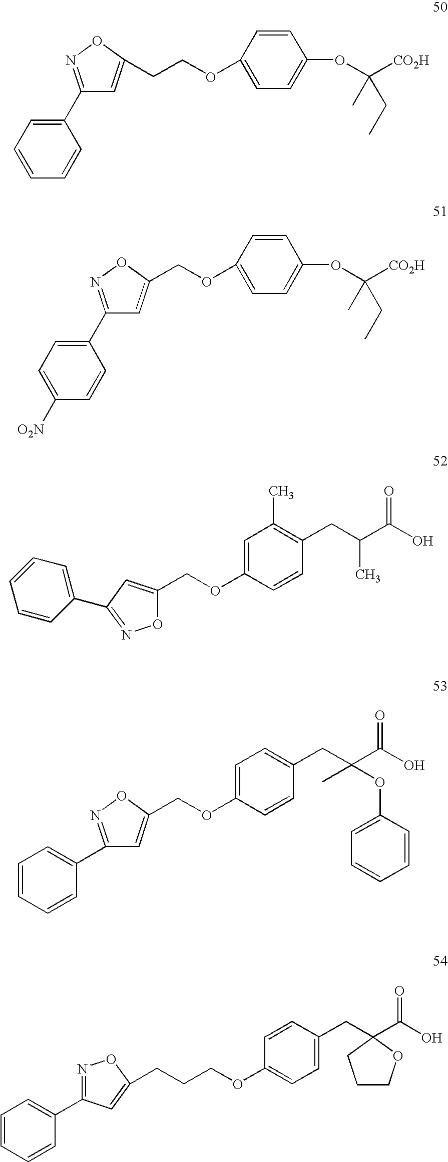

### Example 55

The derivatives described herein are believed to possess at least a baseline level of PPAR agonist activity and as such are useful candidates for use in treating metabolic disorders. Generally, suitable PPAR agonists are believed to be useful for attenuating and/or treatment of diabetic dyslipidemia, metabolic syndrome, diabetes, cardiovascular disease, and obesity. The following procedure was used for in vitro determination of PPARα, γ and δ transactivation.

The ligand binding domain of human PPARγ1, PPARα or PPARδ was fused to the C-terminal end of DNA binding domain of yeast transcription factor GAL4 in eukaryotic expression vector. Using SuperFect (Qiagen), HEK-293 cells were transfected with this plasmid and a reporter plasmid harboring the luciferase gene driven under the control of 5 tandem GLA4 response elements. In some experiments, pAdVantage vector (Promega) was used to enhance luciferase expression. Compounds were added approximately 48 hours after transfection at various concentrations and incubated overnight. The cells were lysed and the luciferase activity was measured using the LucLite kit in Top Count (Packard). Luciferase activity was expressed as fold activation relative to untreated cells in the same experiment.

| | Concentration | | | |
|---|---|---|---|---|
| Example No. | (μM) | PPARα | PPARδ | PPARγ |
| 2 | 1 | 1.2 (1.1)* | — | 1.4 (14.7) |
| | 10 | 1.0 (1.6) | | 2.4 (20) |
| | 50 | 1.0 (5.0) | | 2.4 (22) |
| 5 | 1 | 1.7 (1.2) | 9.0 (68) | 0.9 (17) |
| | 10 | 3.8 (1.7) | 10 (74) | 1.2 (21) |
| | 50 | 6.0 (4.0) | 33 (78) | 6.4 (24) |
| 8 | 1 | 1.3 (1.1) | — | 0.5 (11) |
| | 10 | 2.6 (1.7) | | 1.2 (14.7) |
| | 50 | 3.5 (3.4) | | 4.1 (16) |
| 9 | 1 | 0.8 (1.1) | — | 1.2 (14.7) |
| | 10 | 1.2 (1.6) | | 0.9 (20) |
| | 50 | 1.7 (5.0) | | 2.4 (22) |
| 10 | 1 | 4.0 (0.9) | 7.5 (68) | 0.9 (12.5) |
| | 10 | 5.7 (1.4) | 23 (74) | 3.5 (14) |
| | 50 | 5.1 (4.2) | 47 (78) | 9.5 (18) |
| 11 | 1 | 4.1 (1.6) | — | 2.4 (15.4) |
| | 10 | 4.0 (2.5) | | 2.7 (17) |
| | 50 | 4.0 (5.2) | | 3.6 (20.4) |
| 26 | 1 | 1.8 (0.8) | — | 0.1 (17) |
| | 10 | 5.7 (1.4) | | 1.0 (18.9) |
| | 50 | 5.8 (5.0) | | 5.3 (20.8) |
| 28 | 1 | 1.5 (0.6) | — | 2.0 (15) |
| | 10 | 4.8 (1.6) | | 2.8 (17) |
| | 50 | 5.9 (4.1) | | 12.4 (20) |
| 36 | 1 | 4.1 (1.7) | 0.9 (44) | 4.3 (13.9) |
| | 10 | 4.9 (2.7) | 26.4 (49.9) | 9.8 (20.3) |
| | 50 | 5.4 (6.1) | 23.2 (49.5) | 13.5 (21) |
| 39 | 1 | 1.1 (1.1) | — | 1.8 (11.7) |
| | 10 | 3.9 (2.4) | | 3.9 (12.5) |
| | 50 | 4.7 (4.6) | | 9.0 (13) |

*The values in parentheses represent the PPAR activations of the PPAR agonists Wyeth 14643, Rosiglitazone and GW 501516, respectively, obtained at given concentrations

### Example 56

Male Swiss albino mice (SAM) were obtained from National Institute Nutrition (NIN, Hyderabad, India) and housed in Dr. Reddy's Laboratories Ltd (DRL) animal house. All these animals were maintained under 12 hour light and dark cycle at 25±1° C. Animals were given standard laboratory chow (NIN, Hyderabad, India) and water, ad libitum. Mice of 20-25 grams body weight range were used. See Olivier, et al., Atherosclerosis, 1988, 70:107-114, incorporated herein by reference in its entirety. The test compounds were administered orally to mice at the doses shown in the following in table for 6 days. Control mice were treated with vehicle 0.25% carboxymethylcellulose (CMC; dose 10 ml/kg). Blood samples were collected in fed state 1 hour after drug administration on 0 and 6 days of treatment from the retro-orbital sinus through heparinized capillary tubes containing EDTA. After centrifugation, plasma was separated for plasma triglyceride and total cholesterol measurement. See Wieland, Methods of Enzymatic Analysis, Bergermeyer, ed., 1963, 211-214; Trinder, Ann. Clin. Biochenz., 1969, 6:24-27, incorporated herein by reference in their entireties. Measurement of cholesterol and plasma triglyceride was done using a Vitalab Selectra autoanalyser (Merck). The percent reductions in plasma triglycerides (TG)/total cholesterol were calculated according to the following formula:

$$Percent reduction ( \% ) = [ 1 - \frac{T T / O T}{T C / O C} ] \times 100$$

OC=Zero day control group value
OT=Zero day treated group value
TC=Test day control group value
TT=Test day treated group value.

The following results were obtained:

| | | % reduction |
|---|---|---|
| Example No. | Dose (mg/kg) | in TG |
| 5 | 3 | 75 |
| 8 | 3 | 52 |
| 10 | 3 | 75 |
| 11 | 3 | 65 |
| 26 | 1 | 50 |
| 28 | 3 | 48 |
| 36 | 1 | 26 |
| 37 | 3 | 69 |
| 39 | 3 | 44 |

### Example 57

Male Sprague Dawley rats (NIN stock) were bred in DRL animal house. Animals were maintained under 12 hour light and dark cycle at 25±1° C. Rats of 180-200 gram body weight range were used for the experiment. Animals were made hypercholesterolemic by feeding 2% cholesterol and 1% sodium cholate mixed with standard laboratory chow (NIN) for 6 days. Throughout the experimental period the animals were maintained on the same diet. The test compounds were administered orally to rats at doses shown in the following table for 3 days. Control rats were treated with vehicle alone (0.25% CMC; 10 ml/kg). Blood samples were collected in fed state 1 hour after drug administration on 0 and 3 days of treatment from the retro-orbital sinus through heparinized capillary tubes containing EDTA. After centrifugation, plasma sample was separated for total cholesterol, HDL and plasma triglyceride measurement. Measurement of total cholesterol, HDL and plasma triglyceride was done using a Vitalab Selectra autoanalyser (Merck). HDL was measured using a commercial kit. LDL and VLDL cholesterol were calculated from the data obtained for total cholesterol, HDL and plasma triglyceride.

The percent reductions in plasma triglycerides (TG)/total cholesterol (TC) were calculated as described in Example 55. The levels of LDL and VLDL cholesterol were calculated according to the following formulas:
LDL cholesterol in mg/dl=[Total cholesterol−HDL cholesterol−Triglyceride/5]
VLDL cholesterol in mg/dl=[Total cholesterol−HDL cholesterol−LDL cholesterol]

The following results were obtained:

% Reduction

| Example No. | Dose (mg/kg) | TC | TG | HDL* | LDL |
|---|---|---|---|---|---|
| 11 | 3 | 71 | 55 | 139 | 76 |
| 28 | 3 | 70 | 62 | 132 | — |
| 36 | 3 | 52 | 63 | 128 | 62 |
| 37 | 3 | 69 | 68 | 138 | 75 |

*Values for HDL represent % increase

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

We claim:

1. A derivative, which is a compound and/or a pharmaceutically acceptable salt thereof, wherein said compound has the formula (I): 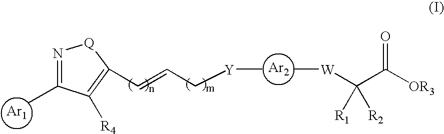
wherein Ar₁ is an optionally substituted aryl or heteroaryl;
Ar₂ is an optionally substituted aryl;
R₁ and R₂, which may be the same or different, are independently hydrogen, hydroxy, halogen or an optionally substituted alkyl, cycloalkyl, aryl, aralkyl, aryloxy, heteroaryl, heterocyclyl or heteroaralkyl, or R₁ and R₂ together form an optionally substituted 5 or 6 membered cyclic ring, which optionally contains one or two hetero atoms selected from O, S or N;
R₃ and R₄, which may be the same or different, are independently hydrogen or an optionally substituted alkyl, cycloalkyl, aryl, aralkyl, heteroaryl, heterocyclyl or heteroaralkyl;
Y is O, S, CH₂ or NR₅, wherein R₅ is hydrogen, alkyl or cycloalkyl;
W is O, S or CH₂;
Q is O or S;
m is 0-6; and
n is 0-1.

2. The derivative of claim 1, wherein Y is O, W is O, Q is O and R₄ is H.

3. The derivative of claim 2, wherein n is 0.

4. The derivative of claim 3, wherein the compound is

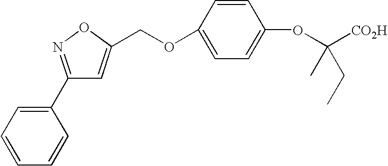

5. The derivative of claim 3, wherein the compound is

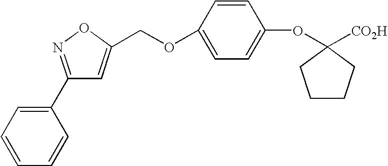

6. The derivative of claim 3, wherein the compound is

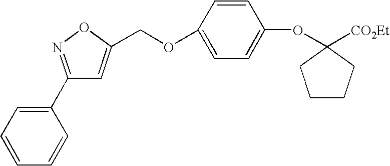

7. The derivative of claim 3, wherein the compound is

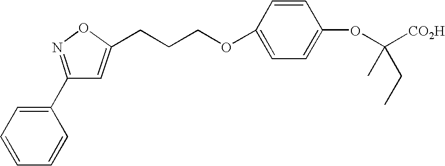

8. The derivative of claim 3, wherein the compound is

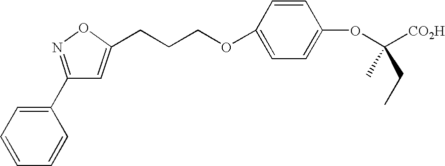

9. The derivative of claim 3, wherein the compound is

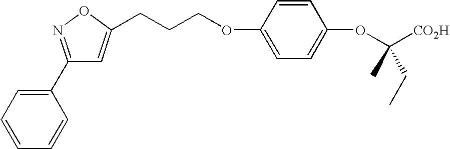

10. The derivative of claim 3, wherein the compound is

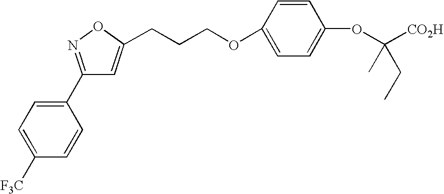

11. The derivative of claim 3, wherein the compound is

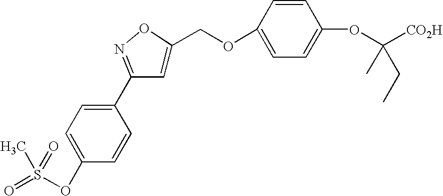

12. The derivative of claim 11, wherein the compound is an arginine salt.

13. The derivative of claim 3, wherein the compound is

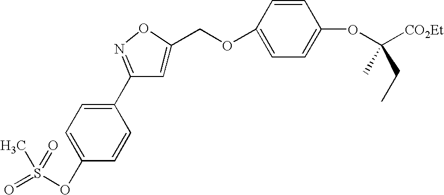

14. The derivative of claim 13, wherein the compound is an arginine salt.

15. The derivative of claim 3, wherein the compound is

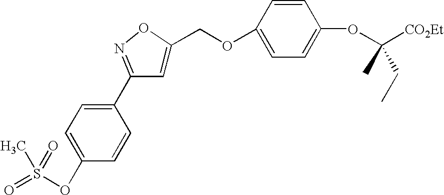

16. The derivative of claim 15, wherein the compound is an arginine salt.

17. The derivative of claim 3, wherein the compound is

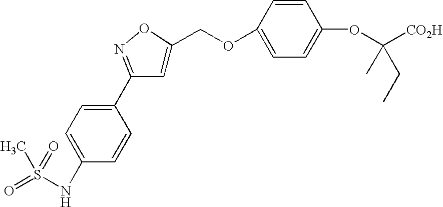

18. The derivative of claim 3, wherein the compound is

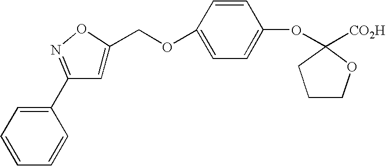

19. The derivative of claim 3, wherein the compound is

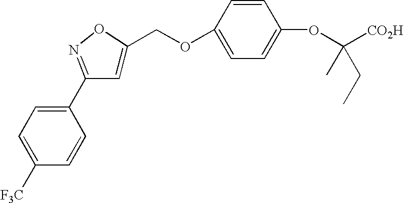

20. The derivative of claim 3, wherein the compound is

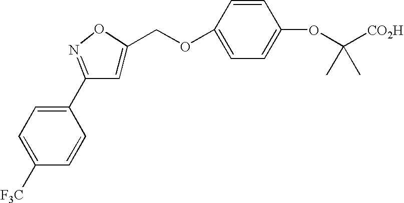

21. The derivative of claim 3, wherein the compound is

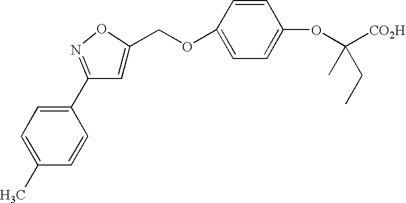

22. The derivative of claim 3, wherein the compound is

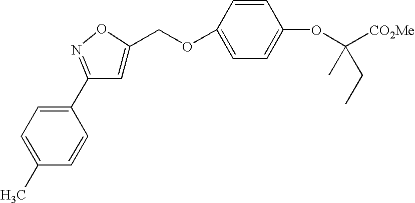

23. The derivative of claim 3, wherein the compound is

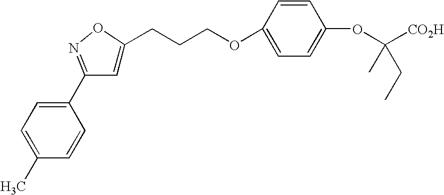

24. The derivative of claim 3, wherein the compound is

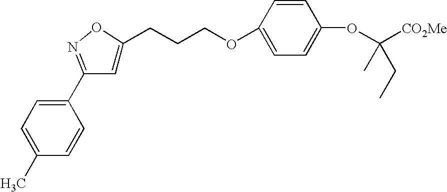

25. The derivative of claim 3, wherein the compound is

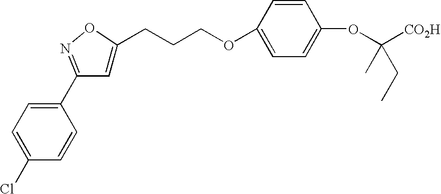

26. The derivative of claim 3, wherein the compound is

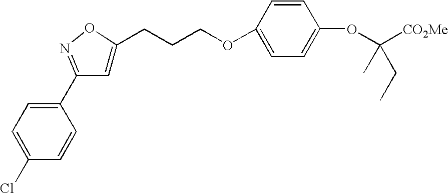

27. The derivative of claim 3, wherein the compound is

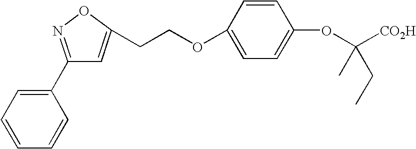

28. The derivative of claim 3, wherein the compound is

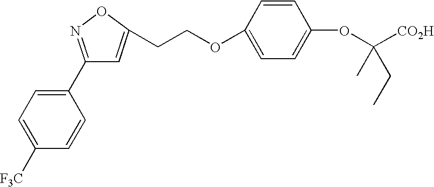

29. The derivative of claim 28, wherein the compound is an arginine salt.

30. The derivative of claim 3, wherein the compound is

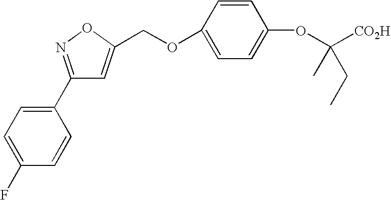

31. The derivative of claim 3, wherein the compound is

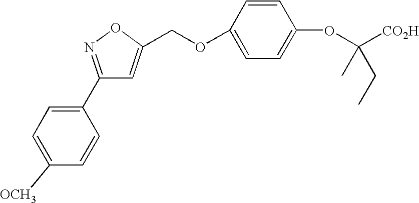

32. The derivative of claim 3, wherein the compound is

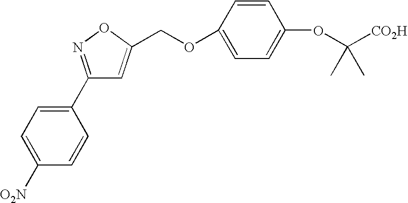

33. The derivative of claim 3, wherein the compound is

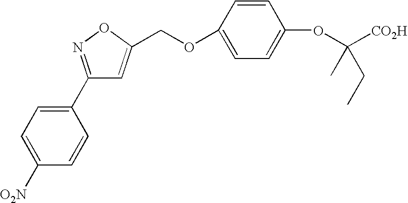

34. The derivative of claim 3, wherein the compound is

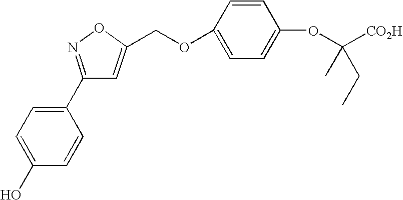

35. The derivative of claim 3, wherein the compound is

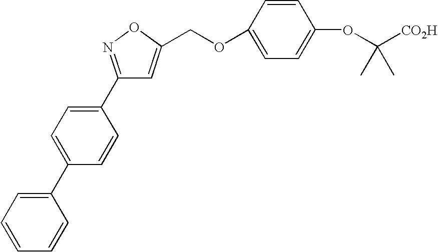

36. The derivative of claim 3, wherein the compound is

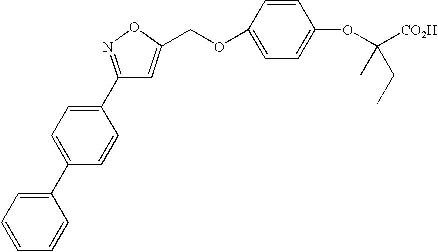

37. The derivative of claim 3, wherein the compound is

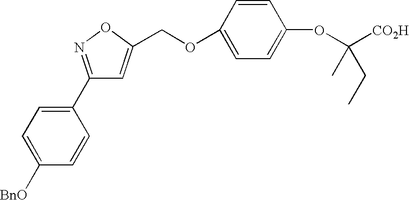

38. The derivative of claim 3, wherein the compound is

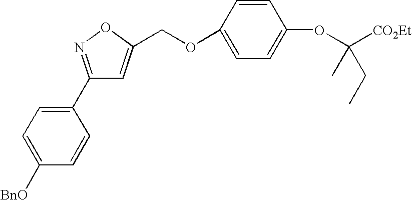

39. The derivative of claim 2, wherein n is 1.

40. The derivative of claim 39, wherein the compound is

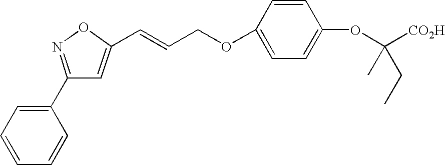

41. The derivative of claim 39, wherein the compound is

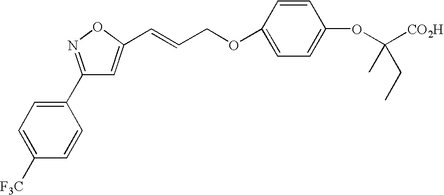

42. The derivative of claim 1, wherein Y is CH₂, W is O, Q is O, R₄ is H and n is 0.

43. The derivative of claim 42, wherein the compound is

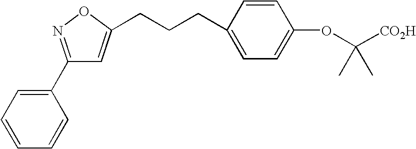

44. The derivative of claim 1, wherein Y is O, W is CH₂, Q is O, R₄ is H and n is 0.

45. The derivative of claim 44, wherein the compound is

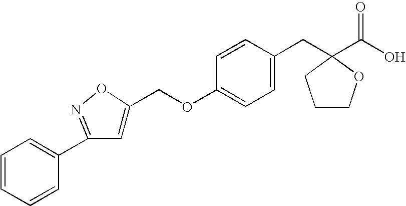

46. The derivative of claim 44, wherein the compound is

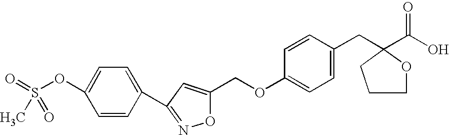

47. The derivative of claim 44, wherein the compound is

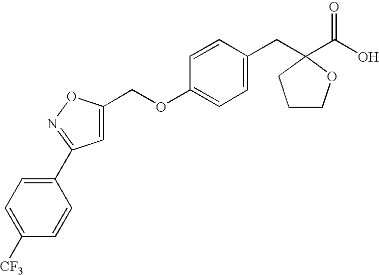

48. The derivative of claim 44, wherein the compound is

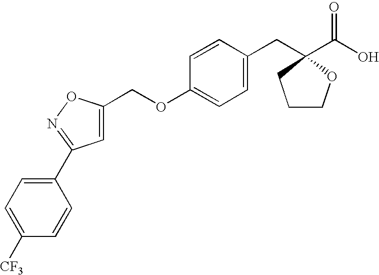

49. The derivative of claim 48, wherein the compound is an arginine salt.

50. The derivative of claim 44, wherein the compound is

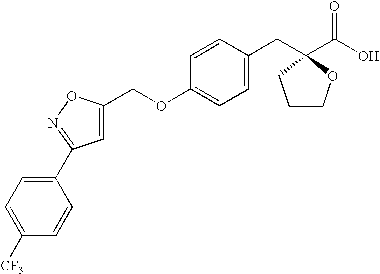

51. The derivative of claim 50, wherein the compound is an arginine salt.

52. The derivative of claim 44, wherein the compound is

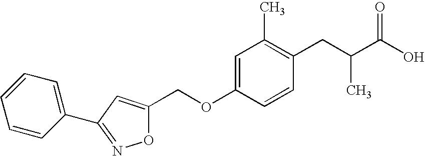

53. The derivative of claim 44, wherein the compound is

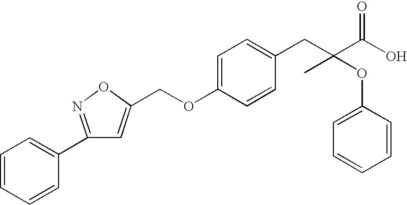

54. The derivative of claim 44, wherein the compound is

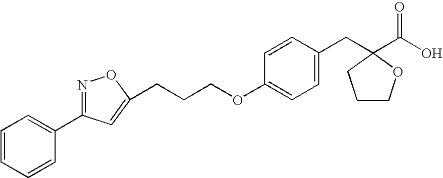

55. The derivative of claim 1, wherein Y is O, W is O, Q is S, R₄ is H and n is 0.

56. The derivative of claim 55, wherein the compound is

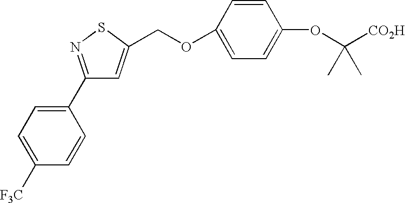

57. The derivative of claim 55, wherein the compound is

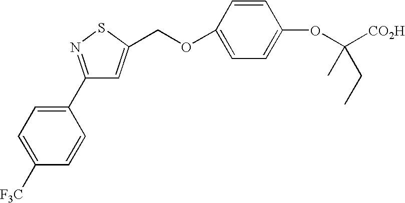

58. The derivative of claim 1, wherein Y is S, W is O, Q is O, R₄ is H and n is 0.

59. The derivative of claim 58, wherein the compound is

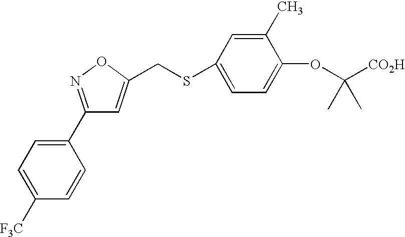

60. The derivative of claim 1, wherein Y is O, W is S, Q is O, R₄ is H and n is 0.

61. The derivative of claim 60, wherein the compound is

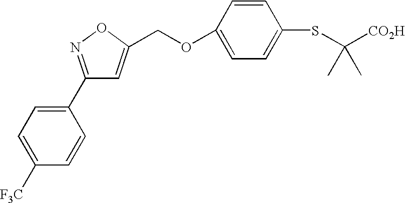

62. The derivative of claim 1, wherein Y is NR₅, W is O, Q is O, R₄ is H, R₅ is H and n is 0.

63. The derivative of claim 62, wherein the compound is

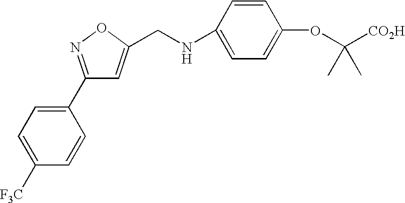

64. The derivative of claim 1, wherein Y is O, W is O, Q is O, R₄ is CH₃ and n is 0.

65. The derivative of claim 64, wherein the compound is

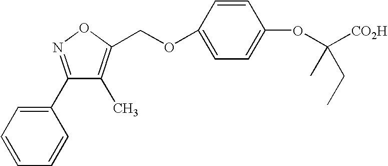

66. A pharmaceutical composition comprising the derivative of claim 1 and one or more pharmaceutically acceptable excipients.

67. A method for producing a PPAR-α agonist activity in an individual in need of such activity comprising administering to said individual a therapeutically effective amount of the pharmaceutical composition of claim 66.

68. A method for treating a disease or disorder in which PPAR-α agonist activity is desired comprising administering to an individual in need of such treatment a therapeutically effective amount of the pharmaceutical composition of claim 66.

69. The method of claim 68, wherein the disease or disorder is selected from the group consisting of diabetes, hypertension, coronary heart disease, atherosclerosis, stroke, peripheral vascular diseases, psoriasis, polycystic ovarian syndrome (PCOS), inflammatory bowel diseases, osteoporosis, myotonic dystrophy, pancreatitis, retinopathy, arteriosclerosis, xanthoma and related disorders.
